(19) United States
(12) Patent Application Publication
(10) Pub. No.: US 2024/0058279 A1
(43) Pub. Date: Feb. 22, 2024

(54) USE OF CELL TURNOVER FACTORS FOR INCREASING TISSUE REGENERATION

(71) Applicant: Flagship Pioneering Innovations V, Inc., Cambridge, MA (US)

(72) Inventors: Peter Joseph Gough, Wayne, PA (US); Darby Schmidt, Arlington, MA (US); Victoria Diane Kutilek, Roslindale, MA (US); Anthony Michael Barsotti, New York, NY (US); Raju Varaha Laxmi N. Pusapati, Fremont, CA (US)

(21) Appl. No.: 18/268,920

(22) PCT Filed: Dec. 21, 2021
(86) PCT No.: PCT/US2021/064710

§ 371 Date: Jun. 21, 2023

Related U.S. Application Data
(60) Provisional application No. 63/128,530, filed on Dec. 21, 2020.

(51) Int. Cl. A61K 31/095; A61P 43/00; C12N 5/00

(52) U.S. Cl. CPC A61K 31/095; A61P 43/00; C12N 5/0018

(57) ABSTRACT

The disclosure provides methods of treating a tissue, organ, organoid or organ culture, the method comprising treating a tissue, organ, organoid or organ culture in vitro with a composition comprising one or more cell turnover factors produced by cells exposed to a stress condition. The disclosure also provides methods of increasing regeneration of a tissue in an organ of a subject, the method comprising administering to the subject a composition comprising one or more cell turnover factors produced by cells exposed to a stress condition, wherein the composition is administered to the subject in an amount sufficient to increase regeneration of the tissue relative to a subject that is not treated with the composition.

### RELATED APPLICATIONS

This application claims priority to U.S. Provisional Application No. 63/128,530, filed on Dec. 21, 2020, the entire contents of which are expressly incorporated herein by reference.

### BACKGROUND

In multicellular organisms, cell death is a critical and active process that is believed to maintain tissue homeostasis and eliminate potentially harmful cells.

### SUMMARY OF THE INVENTION

In certain aspects, the disclosure relates to a method of increasing regeneration of a tissue in an organ of a mammalian subject, the method comprising administering to the subject a composition in an amount and for a time sufficient to increase regeneration of the tissue, wherein the composition is made by (a) exposing mammalian cells to a stress condition, and (b) collecting the exposed cells or conditioned media from the exposed cells to create the composition. In some embodiments, the stress condition is a compound that induces endoplasmic reticlulum stress. In some embodiments, the stress condition is selected from the group consisting of thioacetamide, tunicamycin, PhenolaTi, Zearalenone, Shiga toxin-2, carbon tetrachloride (CCL4) and acetaminophen. In some embodiments, the stress condition is thioacetamide. In some embodiments, the mammalian cells exposed to the stress condition are of a type present in the tissue or organ. In some embodiments, the mammalian cells exposed to the stress condition are selected from the group consisting of liver cells, kidney cells, pancreatic cells, muscle cells, bone cells, cells of the intestinal lining, cardiac cells, lung cells, skin cells, neurons, cells of the central nervous system (CNS), epithelial cells, endothelial cells, fibroblasts, and immune cells. In some embodiments, the mammalian cells exposed to the stress condition are liver cells and the organ is liver. In some embodiments, the cells or the conditioned media are further fractioned to create the composition. In some embodiments, the mammalian cells exposed to the stress condition are autologous to the subject. In some embodiments, the mammalian subject is a human.

In certain aspects, the disclosure relates to a method of increasing regeneration of a tissue in an organ of a subject, the method comprising administering to the subject a composition comprising one or more cell turnover factors produced by cells exposed to a stress condition, wherein the composition is administered to the subject in an amount sufficient to increase regeneration of the tissue relative to a subject that is not treated with the composition. In certain aspects, the disclosure relates to a method of delivering a composition comprising one or more cell turnover factors produced by cells exposed to a stress condition to a subject in need of increased tissue regeneration, the method comprising administering the composition to the subject.

In some embodiments, the tissue is liver tissue and the subject has a disorder selected from the group consisting of chronic liver damage, alcoholic steatohepatitis (ASH), drug-induced liver injury, fulminant and late-onset hepatic failure (LOHF), fulminant hepatitis (FH), liver cirrhosis, liver fibrosis, fulminant hepatic failure (FHF), hepatitis B, and hepatitis C. In some embodiments, the tissue is kidney tissue and the subject has a disorder or condition selected from the group consisting of diabetes mellitus, rheumatoid arthritis, nephritic syndrome, nephrotic syndrome, hypertension nephropathy, polycystic kidney disease, progressive chronic kidney disease, chronic renal failure, Fabry disease, cystinosis, nephronophthisis, Alport's syndrome, reperfusion injury, acute kidney injury, kidney fibrosis and a kidney transplant. In some embodiments, the tissue is pancreatic tissue and the subject has a disorder selected from the group consisting of pancreatic cancer, diabetes mellitus, insulin resistance, hypoglycemia, hyperglycemia, lipase deficiency, cholecystokinin (CCK) deficiency, acute pancreatitis, chronic pancreatitis and hereditary pancreatitis. In some embodiments, the tissue is intestinal lining tissue and the subject has a disorder or condition selected from the group consisting of an inflammatory gastrointestinal disorder, Crohn's disease, inflammatory bowel disease (IBD), diverticulitis, parasitic infection, bacterial infection, a functional gastrointestinal disorder, ulcerative colitis (UC), and a surgical resection of the intestines. In some embodiments, the tissue is cardiac tissue and the subject has a cardiovascular disease selected from the group consisting of a myocardial infarction, heart failure, heart injury by ischemic event, and heart injury by a non-ischemic event. In some embodiments, the tissue is lung tissue and the subject has a disorder or condition selected from the group consisting of acute respiratory distress syndrome (ARDS), chronic obstructive pulmonary disease (COPD), lung cancer, bronchiolitis obliterans organizing pneumonia (BOOP), Coronavirus Disease 2019 (COVID-19), mesothelioma, cystic fibrosis, asthma, idiopathic pulmonary fibrosis, lung failure due to aging, pulmonary fibrosis, interstitial lung disease (ILD), pulmonary arterial hypertension and al-antitrypsin disorder. In some embodiments, the tissue is muscle tissue and the subject has a disorder or condition selected from the group consisting of myositis, autoimmune disease, amyotrophic lateral sclerosis (ALS), sarcopenia, pediatric Charcot-Marie-Tooth disease, muscle loss due to aging, muscle strain from injury, muscle atrophy, muscular dystrophy, dermatomyositis, Guillain-Barré syndrome, multiple sclerosis, polio and polymyositis.

In some embodiments, the composition is formulated to target an organ in which tissue regeneration is increased. In some embodiments, the composition is formulated to target liver, kidney, pancreas, muscle, intestinal lining, heart, or lung. In some embodiments, the composition is formulated to target liver. In some embodiments, the composition is formulated to target kidney. In some embodiments, the composition is administered to the subject in an amount sufficient to decrease necrosis in the tissue in which the tissue regeneration is increased, relative to a subject that is not treated with the composition.

In some embodiments, the composition is administered to the subject in an amount sufficient to decrease steatosis in the tissue in which the tissue regeneration is increased, relative to a subject that is not treated with the composition. In some embodiments, the composition is administered to the subject in an amount sufficient to increase weight of the organ in which the tissue regeneration is increased, relative to a subject that is not treated with the composition. In some embodiments, the composition is administered to the subject in an amount sufficient to increase expression of one or more cell proliferation marker proteins in the tissue, relative to a subject that is not treated with the composition.

In certain aspects, the disclosure relates to a method of increasing tissue regeneration in a transplanted organ or tissue, the method comprising treating an organ or tissue ex vivo with a composition comprising one or more cell turnover factors produced by cells exposed to a stress condition, wherein the organ or tissue is treated with the composition in an amount sufficient to increase tissue regeneration in the organ or tissue after transplantation to a subject relative to an organ or tissue that is not treated with the composition.

In certain aspects, the disclosure relates to a method of treating a tissue, an organ, organoid or organ culture, the method comprising contacting a tissue, an organ, organoid or organ culture in vitro with a composition comprising one or more cell turnover factors produced by cells exposed to a stress condition.

In certain aspects, the disclosure relates to a method of preparing an organ or tissue for transplantation to a subject, the method comprising treating an organ or tissue ex vivo with a composition comprising one or more cell turnover factors produced by cells exposed to a stress condition.

In some embodiments, the organ or tissue is treated with the composition in an amount sufficient to increase tissue regeneration in the organ or tissue after transplantation to the subject relative to an organ or tissue that is not treated with the composition. In some embodiments, the organ or tissue is treated with the composition in an amount sufficient to increase survival of the subject after transplantation of the organ or tissue to the subject relative to a subject transplanted with an organ or tissue that is not treated with the composition. In some embodiments, the organ or tissue is treated with the composition in an amount sufficient to improve engraftment of the organ or tissue after transplantation to the subject relative to an organ or tissue that is not treated with the composition. In some embodiments, the organ or tissue is treated with the composition in an amount sufficient to prolong viability of the organ or tissue before transplantation to the subject relative to an organ or tissue that is not treated with the composition. In some embodiments, the stress condition comprises an abiotic stress, a biotic stress and/or a chemical stress. In some embodiments, the stress condition comprises an abiotic stress. In some embodiments, the abiotic stress is selected from the group consisting of nutrient deprivation, heat, cold, radiation, hypoxia, osmotic pressure, and pH stress. In some embodiments, the stress condition comprises a biotic stress. In some embodiments, the biotic stress is selected from the group consisting of a viral infection with a naturally occurring virus, a bacterial infection, and a fungal infection. In some embodiments, the stress condition induces the cells to undergo apoptosis. In some embodiments, the stress condition comprises a chemical stress. In some embodiments, the chemical stress comprises contacting the cells with a compound that promotes production of cell turnover factors by the cells. In some embodiments, the cells exposed to a stress condition are allogeneic to the subject. In some embodiments, the cells exposed to a stress condition are autologous to the subject. In some embodiments, the cells exposed to a stress condition are selected from the group consisting of liver cells, kidney cells, pancreatic cells, muscle cells, bone cells, cells of the intestinal lining, cardiac cells, lung cells, skin cells, neurons, cells of the central nervous system (CNS), epithelial cells, endothelial cells, fibroblasts, and immune cells. In some embodiments, the epithelial cells, endothelial cells, fibroblasts or immune cells are from the liver, kidney, pancreas, muscle, bone, intestinal lining, heart, lung, skin, or the central nervous system. In some embodiments, the cells exposed to a stress condition are from the same type of tissue in which the tissue regeneration occurs. In some embodiments, the cells exposed to a stress condition are liver cells, and the tissue in which the tissue regeneration occurs is liver tissue; the cells exposed to a stress condition are kidney cells and the tissue in which the tissue regeneration occurs is kidney tissue; the cells exposed to a stress condition are lung cells and the tissue in which the tissue regeneration occurs is lung tissue; the cells exposed to a stress condition are muscle cells and the tissue in which the tissue regeneration occurs is muscle tissue; the cells exposed to a stress condition are bone cells and the tissue in which the tissue regeneration occurs is bone tissue; the cells exposed to a stress condition are pancreatic cells and the tissue in which the tissue regeneration occurs is pancreatic tissue; the cells exposed to a stress condition are cardiac cells and the tissue in which the tissue regeneration occurs is cardiac tissue; the cells exposed to a stress condition are cells of the intestinal lining and the tissue in which the tissue regeneration occurs is the lining of the intestine; the cells exposed to a stress condition are skin cells and the tissue in which the tissue regeneration occurs is skin tissue; the cells exposed to a stress condition are cells of the CNS and the tissue in which the tissue regeneration occurs is CNS tissue; the cells exposed to a stress condition are epithelial cells and the tissue in which the tissue regeneration occurs is epithelium; or the cells exposed to a stress condition are endothelial cells and the tissue in which the tissue regeneration occurs is endothelium. In some embodiments, the cells exposed to a stress condition are not from the same type of tissue in which the tissue regeneration occurs. In some embodiments, the cells exposed to a stress condition are cancer cells. In some embodiments, the cancer cells are immortalized. In some embodiments, the cancer cells are primary cells isolated from a subject. In some embodiments, the composition does not comprise intact cells. In some embodiments, the composition comprises a cell-free extract prepared from the cells exposed to a stress condition. In some embodiments, the composition comprises conditioned media from the cells exposed to a stress condition. In some embodiments, the composition comprises a functional fraction of the conditioned media. In some embodiments, the functional fraction is prepared by isolating molecules based on molecular weight from the conditioned media. In some embodiments, the composition comprises one or more cell turnover factors isolated from the cells. In some embodiments, the one or more cell turnover factors are purified or partially purified. In some embodiments, the composition comprises at least ten cell turnover factors. In some embodiments, the composition comprises at least two cell turnover factors. In some embodiments, the composition comprises only one cell turnover factor. In some embodiments, the organ is a solid organ. In some embodiments, the organ is selected from liver, kidney, pancreas, muscle, intestinal lining, heart, and lung. In some embodiments, the organ has an injury. In some embodiments, the injury is caused by a drug, a toxin, viral infection, or surgery to the organ. In some embodiments, the composition is administered to the subject after the organ injury. In some embodiments, the subject is in need of surgery to the organ. In some embodiments, the composition is administered to the subject before surgery to the organ.

In some embodiments, the composition is administered to the subject after surgery to the organ. In some embodiments, the surgery comprises cancer resection involving the organ. In some embodiments, the tissue is selected from the group consisting of liver tissue, kidney tissue, pancreas tissue, muscle tissue, intestinal lining, cardiac tissue and lung tissue. In some embodiments, the method further comprises a step of preparing the composition comprising the one or more cell turnover factors. In some embodiments, the step of preparing the composition comprises exposing the cells to the stress condition.

In certain aspects, the disclosure relates to a method of increasing regeneration of a tissue in an organ of a subject, the method comprising administering to the subject a composition comprising a compound that induces cell turnover of a target cell in the subject, wherein the composition is administered in an amount sufficient to increase regeneration of the tissue relative to a subject that is not treated with the composition. In some embodiments, the compound induces endoplasmic reticulum (ER) stress in the target cell. In some embodiments, the ER stress comprises an unfolded protein response. In some embodiments, the compound induces apoptosis in the target cell. In some embodiments, the compound induces production of reactive oxygen species (ROS) in the target cell. In some embodiments, the production of ROS in the target cell is at a level sufficient to induce death of the target cell. In some embodiments, the compound is a small molecule. In some embodiments, the compound is a protein. In some embodiments, the compound is targeted to the organ in which tissue regeneration is increased. In some embodiments, the compound is targeted to liver, kidney, pancreas, muscle, bone, intestinal lining, heart, or lung. In some embodiments, the compound is conjugated to an antibody targeted to the organ in which tissue regeneration is increased.

In some embodiments, the antibody specifically binds to a tissue-specific antigen of the organ in which tissue regeneration is increased. In some embodiments, the antibody is targeted to liver. In some embodiments, the compound is a toxin. In some embodiments, the compound is selected from the group consisting of thioacetamide, tunicamycin, PhenolaTi, Zearalenone, Shiga toxin-2, carbon tetrachloride (CCL4) and acetaminophen. In some embodiments, the compound is a chemotherapeutic agent. In some embodiments, the compound is not a compound that induces iron-dependent cellular disassembly. In some embodiments, the target cell is a liver cell and the tissue is liver tissue; the target cell is a kidney cell and the tissue is kidney tissue; the target cell is a lung cell and the tissue is lung tissue; the target cell is a muscle cell and the tissue is muscle tissue; the target cell is a bone cell and the tissue is bone tissue; the target cell is a pancreatic cell and the tissue is pancreatic tissue; the target cell is a cardiac cell and the tissue is cardiac tissue; the target cell is a cell of the intestinal lining and the tissue is the lining of the intestine; the target cell is a skin cell and the tissue is skin tissue; the target cell is a cell of the CNS and the tissue is CNS tissue; the target cell is an epithelial cell and the tissue is epithelium; or the target cell is an endothelial cell and the tissue is endothelium. In some embodiments, the target cell is in the organ or tissue in which tissue regeneration is increased. In some embodiments, the target cell is in a tissue adjacent to the organ or tissue in which tissue regeneration is increased. In some embodiments, the target cell is selected from the group consisting of an epithelial cell, a fibroblast, an endothelial cell and an immune cell. In some embodiments, the immune cell is a monocyte or a macrophage.

### BRIEF DESCRIPTION OF THE DRAWINGS

FIG. 1 shows the dosing and sample collection schedule for the mouse partial hepatectomy liver regeneration study described in Example 1.

FIGS. 2A and 2B show the liver weight on Day 2 and Day 4, respectively, of sham mice or mice treated with conditioned medium (CM) or HGF plasmid. The columns from left to right are sham, CM0, HGF plasmid, CM1, CM2, CM3, CMS, CM6 and CM7.

FIGS. 3A and 3B show the liver index on Day 2 and Day 4, respectively, of sham mice or mice treated with conditioned medium (CM) or HGF plasmid. The columns from left to right are sham, CM0, HGF plasmid, CM1, CM2, CM3, CMS, CM6 and CM7.

FIG. 4 shows the HGF concentration in plasma of sham mice or mice treated with CM0 or HGF plasmid. The columns from left to right on each day are sham, CM0 and HGF plasmid.

FIG. 5 shows the steatosis score on Day 2 of sham mice or mice treated with CM or HGF plasmid. The columns from left to right are sham, CM0, HGF plasmid, CM1, CM2, CM3, CM5, CM6 and CM7.

FIGS. 6A and 6B show necrosis score on Day 2 and Day 4, respectively, of sham mice or mice treated with CM or HGF plasmid. The columns from left to right are sham, CM0, HGF plasmid, CM1, CM2, CM3, CM5, CM6 and CM7.

FIG. 7 shows representative images of H&E staining of sham mice or mice treated with CM or HGF plasmid.

FIGS. 8A and 8B show PCNA on Day 2 and Day 4, respectively, of sham mice or mice treated with CM or HGF plasmid. The columns from left to right are sham, CM0, HGF plasmid, CM1, CM2, CM3, CMS, CM6 and CM7.

FIG. 9 shows representative images of PCNA staining of sham mice or mice treated with CM or HGF plasmid. The arrows indicate positive cells.

FIGS. 10A and 10B show Ki67 levels on Day2 and Day 4, respectively, of sham mice or mice treated with CM or HGF plasmid. The columns from left to right are sham, CM0, HGF plasmid, CM1, CM2, CM3, CMS, CM6 and CM7.

FIG. 11 shows representative images of PCNA staining of sham mice or mice treated with CM or HGF plasmid. The arrows indicate positive cells.

### DETAILED DESCRIPTION

The present disclosure relates to a method of increasing regeneration of a tissue in an organ of a subject, the method comprising administering to the subject a composition comprising one or more cell turnover factors produced by cells exposed to a stress condition, wherein the composition is administered to the subject in an amount sufficient to increase regeneration of the tissue relative to a subject that is not treated with the composition. The present disclosure also relates to a method of treating a tissue, an organ, organoid or organ culture, the method comprising treating a tissue, an organ (e.g. an organ for transplantation), organoid or organ culture in vitro with a composition comprising one or more cell turnover factors produced by cells exposed to a stress condition.

Applicants have surprisingly shown that cell turnover factors produced by cells exposed to a stress condition increase tissue regeneration in a mouse model of liver injury.

Accordingly, administration of cell turnover factors produced by cells exposed to a stress condition may be used to treat disorders that would benefit from increased tissue regeneration.

### I. Definitions

The term “abiotic stress condition” as used herein refers to any stress condition that does not comprise contacting cells with a virus, bacterium, fungus or other living organism. Suitable abiotic stress conditions include, but are not limited to, environmental stress conditions and chemical stress conditions, as defined herein.

The term “biotic stress condition” as used herein refers to a stress condition that comprises contacting cells with a virus, bacterium, fungus or other living organism. In some embodiments, a biotic stress condition comprises infecting cells with a virus, bacterium, fungus or other living organism.

The term “chemical stress condition” as used herein refers to a stress condition that comprises contacting cells with a compound that induces production of cell turnover factors by the cells. Suitable compounds include, but are not limited to, small molecules, nucleic acids and proteins.

The term “environmental stress condition” as used herein refers to a stress condition that comprises exposing cells to an environment that induces production of cell turnover factors by the cells. Suitable environmental stress conditions include, but are not limited to, nutrient deprivation, heat, cold, radiation, hypoxia, osmotic pressure, and pH stress.

The terms “administer”, “administering” or “administration” include any method of delivery of a pharmaceutical composition or agent into a subject's system or to a particular region in or on a subject.

As used herein, “administering in combination”, “co-administration” or “combination therapy” is understood as administration of two or more active agents using separate formulations or a single pharmaceutical formulation, or consecutive administration in any order such that, there is a time period while both (or all) active agents overlap in exerting their biological activities. It is contemplated herein that one active agent (e.g., a cell turnover factor) can improve the activity of a second therapeutic agent, for example, can sensitize target cells, to the activities of the second therapeutic agent or can have a synergistic effect with the second therapeutic agent. “Administering in combination” does not require that the agents are administered at the same time, at the same frequency, or by the same route of administration. As used herein, “administering in combination”, “co-administration” or “combination therapy” includes administration of a composition comprising one or more cell turnover factors produced by cells exposed to a stress condition with one or more additional therapeutic agents.

“Cell turnover”, as used herein, refers to a dynamic process that reorders and disseminates the material within a cell and may ultimately result in cell death. Cell turnover includes the production and release from the cell of cell turnover factors.

“Cell turnover factors”, as used herein, are molecules and cell fragments produced by a cell undergoing cell turnover that are ultimately released from the cell and influence the biological activity of other cells. Cell turnover factors can include proteins, peptides, carbohydrates, lipids, nucleic acids, small molecules, and cell fragments (e.g. vesicles and cell membrane fragments).

A “cell turnover pathway gene”, as used herein, refers to a gene encoding a polypeptide that promotes, induces, or otherwise contributes to a cell turnover pathway.

“Thanotransmission”, as used herein, is communication between cells that is a result of activation of a cell turnover pathway in a target signaling cell, which signals a responding cell to undergo a biological response. Thanotransmission may be induced in a target signaling cell by modulation of cell turnover pathway genes in said cell through, for example, viral or other gene therapy delivery to the target signaling cell of genes that promote such pathways. The target signaling cell in which a cell turnover pathway has been thus activated may signal a responding cell through factors actively released by the signaling cell, or through intracellular factors of the signaling cell that become exposed to the responding cell during the cell turnover (e.g., cell death) of the signaling cell.

As used herein, the terms “increasing” (or “activating”) and “decreasing” refer to modulating resulting in, respectively, greater or lesser amounts, function or activity of a parameter relative to a reference. For example, subsequent to administration of a preparation described herein, a parameter (e.g. tissue regeneration, cell proliferation) may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration. Generally, the metric is measured subsequent to administration at a time that the administration has had the recited effect, e.g., at least one day, one week, one month, 3 months, 6 months, after a treatment regimen has begun. Similarly, pre-clinical parameters (such as tissue regeneration in a test mammal, by a preparation described herein) may be increased or decreased by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration.

As used herein, a “small molecule” is a molecule that has a molecular weight of less than 1000 Da. In some embodiments, the small molecule has a molecular weight of less than 900, 800, 700, 600 or 500 Da. In certain embodiments, a small molecule does not include a nucleic acid molecule. In certain embodiments, a small molecule does not include a peptide more than three amino acids in length.

A “subject” to be treated by the methods of the invention can mean either a human or non-human animal, preferably a mammal, more preferably a human. In certain embodiments, a subject has a detectable or diagnosed disorder prior to initiation of treatments using the methods of the invention. In embodiments, the subject is a non-human mammal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit).

“Therapeutically effective amount” means the amount of a compound that, when administered to a patient for treating a disorder, is sufficient to effect such treatment for the disorder. When administered for preventing a disorder, the amount is sufficient to avoid or delay onset of the disorder. The “therapeutically effective amount” will vary depending on the compound, the disorder and its severity and the age, weight, etc., of the patient to be treated. A therapeutically effective amount need not be curative. A therapeutically effective amount need not prevent a disorder or condition from ever occurring. Instead a therapeutically effective amount is an amount that will at least delay or reduce the onset, severity, or progression of a disease or condition.

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy).

### II. Cell Turnover and Production of Cell Turnover Factors

Cell turnover is a dynamic process that re-orders and disseminates the material within a cell, and which results in the production and release of cell turnover factors that can have a profound effect on the biological activity of other cells. Cell turnover may occur during the process of regulated cell death and is controlled by multiple molecular mechanisms.

Different types of cell turnover result in the production of different cell turnover factors and thereby mediate different biological effects, e.g. tissue regeneration. For example, Applicants have surprisingly shown that exposure of a cell to a stress condition (e.g. treatment with thioacetamide) can result in the production of cell turnover factors that increase tissue regeneration in vivo.

Cell turnover pathways that may result in the production and release of cell turnover factors that increase tissue regeneration include, but are not limited to, necroptosis (e.g. mitochondrial permeability transition (MPT)-driven necroptosis), apoptosis (e.g. extrinsic apoptosis or intrinsic apoptosis), ferroptosis, pyroptosis, NETotic cell death, ETotic cell death, Entotic cell death, parthanotos, inflammatory cell death, autophagy-dependent cell death, lysosome-dependent cell death, and oxeiptosis and combinations thereof, as described below.

Necroptosis

In certain embodiments, exposure of cells to a stress condition induces necroptosis (e.g. MPT-driven necroptosis) in the cells. The term “necroptosis” as used herein refers to

Receptor interacting protein kinase 3 (RIP1- and/or RIPK3)/Mixed lineage kinase-like (MLKL) -dependent necrosis. Several insults cause the inner mitochondrial membrane to abruptly lose osmotic homeostasis, initiating a regulated variant of necroptosis known as mitochondrial permeability transition (MPT)-driven necroptosis. Several triggers can induce necroptosis, including alkylating DNA damage, excitotoxins and the ligation of death receptors. For example, when caspases (and in particular caspase-8 or caspase-10) are inhibited by genetic manipulations (e.g., by gene knockout or RNA interference, RNAi) or blocked by pharmacological agents (e.g., chemical caspase inhibitors), RIPK3 phosphorylates MLKL leading to MLKL assembly into a membrane pore that ultimately activates the execution of necrotic cell death. See Galluzzi et al., 2018, Cell Death Differ. Mar; 25(3): 486-541, incorporated by reference herein in its entirety.

Several methods are known in the art and may be employed for identifying cells undergoing necroptosis and distinguishing from other types of cell turnover and/or cell death through detection of particular markers. These include phosphorylation of RIPK1, RIPK3, and MLKL by antibodies that detect these post-translational modifications, typically by immunoblot or immunostaining of cells. Necroptosis can be distinguished from apoptosis and pyroptosis by the absence of caspase activation, rapid membrane permeabilization, MLKL relocalization to membranes, accumulation of RIPK3 and MLKL into detergent insoluble fractions, RIPK3/MLKL complex formation, and MLKL oligomerization. Necroptosis can be genetically and pharmacologically defined by requirement of both RIPK3 and MLKL as well as their activation.

Apoptosis

In certain embodiments, exposure of cells to a stress condition induces apoptosis in the cells. Apoptosis is a caspase-driven process of programmed cell death that results in DNA fragmentation, degradation of cytoskeletal and nuclear proteins, cross-linking of proteins, formation of apoptotic bodies, expression of ligands for phagocytic cell receptors, and uptake by phagocytic cells.

In certain embodiments, exposure of cells to a stress condition induces extrinsic apoptosis in the cells. The term ‘extrinsic apoptosis’ as used herein refers to instances of apoptotic cell death that are induced by extracellular stress signals which are sensed and propagated by specific transmembrane receptors. Extrinsic apoptosis can be initiated by the binding of ligands, such as FAS/CD95 ligand (FASL/CD95L), tumor necrosis factor a (TNFα), and TNF (ligand) superfamily, member 10 (TNFSF10, best known as TNF-related apoptosis inducing ligand, TRAIL), to various death receptors (i.e., FAS/CD95, TNFα receptor 1 (TNFR1), and TRAIL receptor (TRAILR)1-2, respectively). Alternatively, an extrinsic pro-apoptotic signal can be dispatched by the so-called ‘dependence receptors’, including netrin receptors (e.g., UNCSA-D and deleted in colorectal carcinoma, DCC), which only exert lethal functions when the concentration of their specific ligands falls below a critical threshold level. See Galluzzi et al., 2018, Cell Death Differ. Mar; 25(3): 486-541, incorporated by reference herein in its entirety.

Several methods are known in the art and may be employed for identifying cells undergoing apoptosis and distinguishing from other types of cell turnover and/or cell death through detection of particular markers. Apoptosis requires caspase activation and can be suppressed by inhibitors of caspase activation and/or prevention of death by the absence of caspases such as caspase-8 or caspase-9. Caspase activation systematically dismantles the cell by cleavage of specific substrates such as PARP and DFF45 as well as over 600 additional proteins. Apoptotic cell membranes initially remain intact with externalization of phosphotidyl-serine and concomitant membrane blebbing. Mitochondrial outer membranes are typically disrupted releasing into the cytosol proteins such as CytoC and HTRA2. Nuclear DNA is cleaved into discrete fragments that can be detected by assays known in the art.

Ferroptosis

In certain embodiments, exposure of cells to a stress condition induces ferroptosis in the cells. The term “Ferroptosis”, as used herein, refers to a process of regulated cell death that is iron dependent and involves the production of reactive oxygen species. In some embodiments, ferroptosis involves the iron-dependent accumulation of lipid hydroperoxides to lethal levels. The sensitivity to ferroptosis is tightly linked to numerous biological processes, including amino acid, iron, and polyunsaturated fatty acid metabolism, and the biosynthesis of glutathione, phospholipids, NADPH, and Coenzyme Q10. Ferroptosis involves metabolic dysfunction that results in the production of both cytosolic and lipid ROS, independent of mitochondria but dependent on NADPH oxidases in some cell contexts (See, e.g., Dixon et al., 2012, Cell 149(5):1060-72, incorporated by reference herein in its entirety).

Several methods are known in the art and may be employed for identifying cells undergoing ferroptosis and distinguishing from other types of cell turnover and/or cell death through detection of particular markers. (See, for example, Stockwell et al., 2017, Cell 171: 273-285, incorporated by reference herein in its entirety). For example, because ferroptosis may result from lethal lipid peroxidation, measuring lipid peroxidation provides one method of identifying cells undergoing iron-dependent cellular disassembly. C11-BODIPY and Liperfluo are lipophilic ROS sensors that provide a rapid, indirect means to detect lipid ROS (Dixon et al., 2012, Cell 149: 1060-1072). Liquid chromatography (LC)/tandem mass spectrometry (MS) analysis can also be used to detect specific oxidized lipids directly (Friedmann Angeli et al., 2014, Nat. Cell Biol. 16: 1180-1191; Kagan et al., 2017, Nat. Chem. Biol. 13: 81-90). Isoprostanes and malondialdehyde (MDA) may also be used to measure lipid peroxidation (Milne et al., 2007, Nat. Protoc. 2: 221-226; Wang et al., 2017, Hepatology 66(2): 449-465). Kits for measuring MDA are commercially available (Beyotime, Haimen, China).

Other useful assays for studying ferroptosis include measuring iron abundance and GPX4 activity. Iron abundance can be measured using inductively coupled plasma-MS or calcein AM quenching, as well as other specific iron probes (Hirayama and Nagasawa, 2017, J. Clin. Biochem. Nutr. 60: 39-48; Spangler et al., 2016, Nat. Chem. Biol. 12: 680-685), while GPX4 activity can be detected using phosphatidylcholine hydroperoxide reduction in cell lysates using LC-MS (Yang et al., 2014, Cell 156: 317-331). In addition, ferroptosis may be evaluated by measuring glutathione (GSH) content. GSH may be measured, for example, by using the commercially available GSH-Glo Glutathione Assay (Promega, Madison, WI).

Ferroptosis may also be evaluated by measuring the expression of one or more marker proteins. Suitable marker proteins include, but are not limited to, glutathione peroxidase 4 (GPX4), prostaglandin-endoperoxide synthase 2 (PTGS2), and cyclooxygenase-2 (COX-2). The level of expression of the marker protein or a nucleic acid encoding the marker protein may be determined using suitable techniques known in the art including, but not limited to polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, Western blot analysis, in situ hybridization, array analysis, deoxyribonucleic acid sequencing, restriction fragment length polymorphism analysis, and combinations or sub-combinations thereof.

Pyroptosis

In certain embodiments, exposure of cells to a stress condition induces pyroptosis in the cells. “Pyroptosis” as used herein refers to the inherently inflammatory process of caspase 1-, caspase 4-, or caspase 5-dependent programmed cell death. The most distinctive biochemical feature of pyroptosis is the early, induced proximity-mediated activation of caspase-1. The pyroptotic activation of caspase-1, 4 or 5 can occur in the context of a multiprotein platform known as the inflammasome, which involves NOD-like receptors (NLRs) or other sensors such as the cytosolic DNA sensor absent in melanoma 2 (AIM2) that recruit the adaptor protein ASC that promotes caspase-1 activation. Caspases-4/5 may be directly activated by LPS. In both cases, active caspase-1 catalyzes the proteolytic maturation and release of pyrogenic interleukin-10 (IL-1(3) and IL-18. Moreover, in some (but not all) instances, caspase activation targets GSDM-D to drive membrane rupture and cell death. See Galluzzi et al., 2018, Cell Death Differ. Mar; 25(3): 486-541.

Several methods are known in the art and may be employed for identifying cells undergoing pyroptosis and distinguishing from other types of cell turnover and/or cell death through detection of particular markers. Pyroptosis requires caspase-1, caspase-4, or caspase-activity and is usually accompanied by the processing of the pro-IL-1b and/or pro-IL-18, release of these mature cytokines, and membrane permeabilization by a caspase-1/4/5 cleavage fragment of GSDM-D.

Additional Cell Turnover Pathways

Additional cell turnover pathways that may result in the production and release of cell turnover factors that increase tissue regeneration include NETotic cell death, ETotic cell death, Entotic cell death, parthanotos, inflammatory cell death, autophagy-dependent cell death, lysosome-dependent cell death, and oxeiptosis.

“NETotic cell death” or “NETosis” is a form of cell death involving release of neutrophil extracellular traps (NETs), chromatin structures loaded with antimicrobial molecules that can trap and kill various bacterial, fungal and protozoal pathogens. NET release is one of the first lines of defense against pathogens in vivo. See Remijsen et al., 2011, Cell Death & Differentiation 18: 581-588.

“ETotic cell death” or “ETosis” is a cell death pathway that involves the formation of extracellular traps (ETs) by neutrophils and mast cells, and is an important mechanism in innate immune response. ETs consist of a chromatin-DNA backbone with attached antimicrobial peptides and enzymes that trap and kill microbes. See Wartha et al., 2008, Science Signaling Vol. 1, Issue 21, pp. pe25.

“Entotic cell death” or “Entosis” is a cell death pathway that involves the invasion of a living cell into another cell's cytoplasm. See Overholtzer et al., 2007, Cell 131 (5): 966-979.

“Parthanotos” is a form of programmed cell death caused by the accumulation of poly(ADP-ribose) (PAR) and the nuclear translocation of apoptosis-inducing factor (AIF) from mitochondria. See David et al., 2009, Front Biosci (Landmark Ed). 2009; 14: 1116-1128.

“Oxeiptosis” is a caspase-independent cell death pathway that links the reactive oxygen species (ROS) sensing capacity of KEAP1 to a cell death pathway involving PGAMS and AIFM1. Oxeiptosis is anti-inflammatory when activated by increased intracellular ROS levels and upon pathogen encounter. See Scaturro et al., 2019, Current Opinion in Immunology, Volume 56, February 2019, Pages 37-43.

### Induction of Cell Turnover and Production of Cell Turnover Factors by Stress Conditions

The methods provided herein may involve administering compositions produced by exposing cells to stress conditions that induce cell turnover and production of cell turnover factors. In some embodiments, the stress condition comprises an abiotic stress condition, for example an environmental stress condition or a chemical stress condition. In some embodiments, the stress condition comprises a biotic stress condition, for example, contacting the cells with a virus, bacterium, fungus or other living organism.

In some embodiments, the abiotic stress condition comprises or consists of an environmental stress condition. Environmental stress conditions suitable for carrying out the methods of the invention include, but are not limited to, nutrient deprivation, heat, cold, radiation, hypoxia, osmotic pressure, and pH stress.

Nutrient deprivation suitable for inducing cell turnover and producing cell turnover factors may comprise culturing cells in a medium lacking sufficient nutrients for sustained cell growth, such as Hank's Balanced Salt Solution (HBSS) or phosphate buffered saline (PBS). In one embodiment, the nutrient deprivation comprises or consists of selenium deficiency.

Heat stress conditions suitable for inducing cell turnover and producing cell turnover factors may comprise exposing a cell to a temperature that is at least 1, 2, 3, 4, 5, 6, 7, 8, 9, 10, 11, 12, 13, 14, 15, 16, 17, 18, 10, 20, 30, 40 or 50° C. higher than the optimal cultivation temperature for the cell, e.g. 37° C. Cold stress conditions suitable for inducing cell turnover and producing cell turnover factors may comprise exposing a cell to a temperature that is at least 1, 2, 3, 4, 5, 6, 7, 8, 9, 10, 11, 12, 13, 14, 15, 16, 17, 18, 10, 20, 30, 40 or 50° C. lower than the optimal cultivation temperature for the cell, e.g. 37° C.

Radiation stress conditions suitable for inducing cell turnover and producing cell turnover factors may comprise, for example, UV radiation, gamma radiation, X-rays, infrared radiation or microwaves. Methods of treating cells with radiation are known in the art and are described, for example, in US 2012/0045418, which is incorporated by reference herein in its entirety. Cells may be irradiated with, for example, at least 10, 20, 30, 40 or 50 Gy to induce cellular disassembly. In some embodiments, the stress condition does not comprise radiation. In some embodiments, the stress condition does not comprise one or more of UV radiation, gamma radiation, X-rays, infrared radiation or microwaves.

Hypoxia is a condition in which a cell is deprived of adequate oxygen supply. Hypoxia stress conditions suitable for inducing cell turnover and producing cell turnover factors may comprise exposing a cell to oxygen concentrations that are at least 5%, 10%, 15%, 20%, 25%, 30%, 40%, 50%, 60%, 70%, 80% or 90% lower than the optimal oxygen concentration for the cell.

Osmotic pressure may be increased by the addition of salt (e.g. NaCl) to the culture medium of a cell. Osmotic pressure stress conditions suitable for inducing cell turnover and producing cell turnover factors may comprise exposing a cell to osmotic pressure that is at least 5%, 10%, 15%, 20%, 25%, 30%, 40%, 50%, 60%, 70%, 80%, 90%, 100%, 200%, 300%, 400%, or 500% higher than the optimal osmotic pressure for a cell.

Cell turnover and production of cell turnover factors may also be induced in a cell by exposing the cell to a pH that is higher or lower than the optimal pH for the cell. The pH of the culture medium for the cell may be adjusted by adding acids or bases to the culture medium. In some embodiments, the pH of the culture medium for the cell is at least 5.0, 5.5, 6.0, 6.5, 7.0, 7.2, 7.5 or 8.0. In some embodiments, the pH of the culture medium for the cell is less than 8.0, 7.5, 7.2, 7.0, 6.5, 6.0, 5.5 or 6.0. Any of these values may be used to define a range for the pH of the culture medium. For example, in some embodiments, the pH of the culture medium is 6.5 to 7.2, 7.5 to 8.0, or 6.0 to 6.5.

In some embodiments, the abiotic stress condition comprises or consists of a chemical stress condition. A chemical stress condition comprises contacting cells with a compound that induces cell turnover and promotes production of cell turnover factors by the cells.

Compounds that induce cell turnover and produce cell turnover factors may include small molecules, nucleic acids or proteins. As used herein, a “small molecule” is a molecule that has a molecular weight of less than 1000 Da. In some embodiments, the small molecule has a molecular weight of less than 900, 800, 700, 600 or 500 Da. In certain embodiments, a small molecule does not include a nucleic acid molecule. In certain embodiments, a small molecule does not include a peptide more than three amino acids in length.

Nucleic acids that induce cell turnover may include, but are not limited to, antisense DNA molecules, antisense RNA molecules, double stranded RNA, siRNA, cDNA, or a Clustered Regularly Interspaced Short Palindromic Repeats (CRISPR)—CRISPR associated (Cas) (CRISPR-Cas) system guide RNA. In some embodiments, the nucleic acid encodes a protein that induces cell turnover and release of cell turnover factors when expressed in a cell.

In some embodiments, the nucleic acid induces cell turnover and release of cell turnover factors by inhibiting expression of one or more genes in the cell.

Nucleic acids that induce cell turnover include both single stranded and double stranded (i.e., nucleic acid therapeutics having a complementary region of at least 15 nucleotides in length) nucleic acids that are complementary to a target sequence in a cell. Antisense nucleic acid therapeutic agents are single stranded nucleic acid therapeutics, typically about 16 to 30 nucleotides in length, and are complementary to a target nucleic acid sequence in the cell.

In some embodiments, the nucleic acid that induce cell turnover is a single-stranded antisense RNA molecule. An antisense RNA molecule is complementary to a sequence within a target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. Patents directed to antisense nucleic acids, chemical modifications, and therapeutic uses include, for example: U.S. Pat. No. 5,898,031 related to chemically modified RNA-containing therapeutic compounds; U.S. Pat. No. 6,107,094 related methods of using these compounds as therapeutic agents; U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality of RNA nucleosides and at least one chemical modification. The entire contents of each of the patents listed in this paragraph are incorporated herein by reference.

Nucleic acids that induce cell turnover also include double stranded nucleic acid therapeutics. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims. The RNAi agents that are used in the methods of the invention include agents with chemical modifications as disclosed, for example, in WO/2012/037254, and WO 2009/073809, the entire contents of each of which are incorporated herein by reference.

Proteins that induce cell turnover may include a protein, for example a monoclonal or polyclonal antibody, that inhibits activity of one or more proteins in the cell. The term “antibody”, as used herein, refers to any immunoglobulin (Ig) molecule comprised of four polypeptide chains, two heavy (H) chains and two light (L) chains, or any functional fragment, mutant, variant, or derivation thereof. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass. In some embodiments, the antibody is a full-length antibody. In some embodiments, the antibody is a murine antibody. In some embodiments, the antibody is a human antibody. In some embodiments, the antibody is a humanized antibody. In other embodiments, the antibody is a chimeric antibody. Chimeric and humanized antibodies may be prepared by methods well known to those of skill in the art including CDR grafting approaches (see, e.g., U.S. Pat. Nos. 5,843,708; 6,180,370; 5,693,762; 5,585,089; and 5,530,101), chain shuffling strategies (see, e.g., U.S. Pat. No. 5,565,332; Rader et al. (1998) PROC. NAT'L. ACAD. SCI. USA 95: 8910-8915), molecular modeling strategies (U.S. Pat. No. 5,639,641), and the like.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) NATURE 341: 544-546; and WO 90/05144 A1, the contents of which are herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) SCIENCE 242:423-426; and Huston et al. (1988) PROC. NAT'L. ACAD. SCI. USA 85:5879-5883). Such single chain antibodies are also intended to be encompassed within the term “antigen-binding portion” of an antibody. Other forms of single chain antibodies, such as diabodies are also encompassed. Antigen binding portions can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers found that certain sub-portions within Kabat CDRs adopt nearly identical peptide backbone conformations, despite having great diversity at the level of amino acid sequence (Chothia et al. (1987) J. MOL. BIOL. 196: 901-917, and Chothia et al. (1989) NATURE 342: 877-883). These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139, and MacCallum et al. (1996) J. MOL. BIOL. 262(5): 732-45. Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

In some embodiments, the compound that induces cell turnover is an immunoconjugate or an antibody drug conjugate. The term “immunoconjugate” or “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate. As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

Fab (fragment antigen binding) antibody fragments are immunoreactive polypeptides comprising monovalent antigen-binding domains of an antibody composed of a polypeptide consisting of a heavy chain variable region (V_{H}) and heavy chain constant region 1 (C_{H1}) portion and a poly peptide consisting of a light chain variable (V_{L}) and light chain constant (C_{L}) portion, in which the C_{L} and C_{H1} portions are bound together, preferably by a disulfide bond between Cys residues.

In some embodiments, the compound that induces cell turnover is an antineoplastic agent. Anti-neoplastic agents suitable for use in the methods disclosed herein include, but are not limited to, chemotherapeutic agents (e.g., alkylating agents, such as Altretamine, Busulfan, Carboplatin, Carmustine , Chlorambucil, Cisplatin, Cyclophosphamide, Dacarbazine, Lomustine, Melphalan, Oxaliplatin, Temozolomide, Thiotepa; antimetabolites, such as 5-fluorouracil (5-FU), 6-mercaptopurine (6-MP); Capecitabine (Xeloda®), Cytarabine (Ara-C®), Floxuridine, Fludarabine, Gemcitabine (Gemzar®), Hydroxyurea, Methotrexate, Pemetrexed (Alimta®); anti-tumor antibiotics such as anthracyclines (e.g., Daunorubicin, Doxorubicin (Adriamycin®), Epirubicin, Idarubicin), Actinomycin-D, Bleomycin, Mitomycin-C, Mitoxantrone (also acts as a topoisomerase II inhibitor);

topoisomerase inhibitors, such as Topotecan, Irinotecan (CPT-11), Etoposide (VP-16), Teniposide, Mitoxantrone (also acts as an anti-tumor antibiotic); mitotic inhibitors such as Docetaxel, Estramustine, Ixabepilone, Paclitaxel, Vinblastine, Vincristine, Vinorelbine; corticosteroids such as Prednisone, Methylprednisolone (Solumedrol®), Dexamethasone (Decadron®); enzymes such as L-asparaginase, and bortezomib (Velcade®)). Anti-neoplastic agents also include biologic anti-cancer agents, e.g., anti-TNF antibodies, e.g., adalimumab or infliximab; anti-CD20 antibodies, such as rituximab, anti-VEGF antibodies, such as bevacizumab; anti-HER2 antibodies, such as trastuzumab; anti-RSV, such as palivizumab.

In some embodiments, the compound that induces cell turnover is a toxin, for example, a toxin that induces ER stress and/or an unfolded protein response. Toxins that induces ER stress and/or an unfolded protein response are known in the art and described herein. In some embodiments, the compound that induces cell turnover is not an agent that induces iron-dependent cell turnover, e.g. ferroptosis.

In some embodiments, the stress condition comprises a biotic stress condition. In some embodiments, the biotic stress condition is selected from a viral infection (e.g. with a naturally occurring virus), a bacterial infection, and a fungal infection.

In some embodiments, the virus is selected from the group consisting of Hepatitis A, Hepatitis B, Hepatitis C, human herpes virus 1 (HHV-1), HHV-2, HHV-3, HHV-4, HHV-5, HHV-6, HHV-7, HHV-8, human papillomavirus (HPV), respiratory syncytial virus (RSV), HIV, SARS-CoV-2, Dengue virus, Chikungunya virus, Zika virus, Norwalk virus and West Nile virus.

In some embodiments, the bacterium is selected from the group consisting of Mycobacterium tuberculosis (e.g. multidrug-resistant Mycobacterium tuberculosis (MDR-TB)), Staphylococcus aureus (e.g. methicillin-resistant Staphylococcus aureus (MRSA)), and a bacterium that causes bacterial meningitis. Bacteria that cause bacterial meningitis include, but are not limited to, Streptococcus pneumoniae, Neisseria meningitides, Haemophilus influenza, and Listeria monocytogenes.

The stress conditions described herein may have various effects on the cells that induce the cell to produce cell turnover factors. For example, in some embodiments, the stress condition induces the cells to undergo any of the cell turnover pathways described herein, e.g. necroptosis, apoptosis, ferroptosis, pyroptosis and combinations thereof. In a particular embodiment, the stress condition induces the cell to undergo apoptosis (e.g. extrinsic apoptosis).

In some embodiments, the stress condition (e.g. a compound that induces cell turnover as described herein) induces endoplasmic reticulum (ER) stress in the cells undergoing cell turnover. The ER is an important site for protein folding and maturation in eukaryotes. The cellular requirement to synthesize proteins within the ER is matched by its folding capacity. However, the physiological demands or aberrations in folding may result in an imbalance which can lead to the accumulation of misfolded protein, also known as “ER stress.” ER stress is an important mechanism in the progression of chronic and acute liver diseases, especially in the progression and recovery of liver fibrosis. Excessive and long-term ER stress induces apoptosis, which is considered to be an important pathway in the development of liver fibrosis.

The ER stress may comprise an unfolded protein response. The unfolded protein response (UPR) is a cell-signaling system that readjusts ER folding capacity to restore protein homeostasis in cells undergoing ER stress. See Adams et al., 2019, Mol. Biosci. 6(11):1-12.

In some embodiments, the stress condition (e.g. a compound that induces cell turnover as described herein) induces an unfolded protein response in the cells undergoing cell turnover.

In some embodiments, the stress condition induces production of reactive oxygen species (ROS) in the cell undergoing cell turnover. In some embodiments, the production of ROS in the target cell is at a level sufficient to induce death of the cell undergoing cell turnover.

Compounds that induce ER stress and/or an unfolded protein response include, but are not limited to thioacetamide, tunicamycin, PhenolaTi, Zearalenone, Shiga toxin-2, carbon tetrachloride (CCL4) and acetaminophen. In a particular embodiment, the agent that induces cell turnover is thioacetamide.

Thioacetamide is an organosulfur compound (C₂H₅NS) that induces cirrhosis of the liver and ER stress in mouse models. See Su et al., 2020, World J Gastroenterol. July 28; 26(28): 4094-4107.

Tunicamycin is a mixture of homologous nucleoside antibiotics that inhibits the UDP-HexNAc: polyprenol-P HexNAc-1-P family of enzymes. In eukaryotes, this includes the enzyme GlcNAc phosphotransferase (GPT), which catalyzes the transfer of N-acetylglucosamine-1-phosphate from UDP-N-acetylglucosamine to dolichol phosphate in the first step of glycoprotein synthesis. Tunicamycin blocks N-linked glycosylation (N-glycans) and treatment of cultured human cells with tunicamycin causes cell cycle arrest in G1 phase. It has also been shown to induce unfolded protein response. See Chan et al., 2005, FASEB journal, Volume19, Issue11: 1510-1512.

PhenolaTi is a titanium (IV)-based compound (bis(phenolato)bis(alkoxo)Ti(IV)) that is used as a non-toxic anti-cancer drug. PhenolaTi induces apoptosis and cell-cycle arrest at the G2/M phase in MCF7 breast cancer cells, and gene expression studies support the ER as a putative cellular target for PhenolaTi. See Miller et all., 2020, iScience 23(7):101262. PhenolaTi has the following structure:

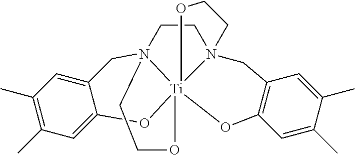

Selenium deficiency has been shown to aggravate toxin-induced injury of rat cardiomyocytes by initiating more aggressive ER stress. See Xu et al., 2018, Chem Biol Interact. 285:96-105.

Zearalenone (ZEA) is a mycotoxin from Fusarium species commonly found in many food commodities and known to exert estrogenic activities which can cause reproductive dysfunction. Zearalenone has been shown to alter cytoskeletal structure via an ER stress-autophagy-oxidative stress pathway in mice. See Zheng et al., 2020, Sci Rep. 8(1): 3320, published correction appears in Sci Rep. 2020 Jun. 25;10(1):10658.

Shiga toxin-2 is produced by enterohemorrhagic Escherichia coli and are responsible for induction of ER stress and kidney injury. The A subunit of the toxin injures the eukaryotic ribosome, and halts protein synthesis in target cells. The endoplasmic reticulum (ER) stress response is hypothesized to induce apoptosis contributing to organ injury. See Parello et al., 2015, Toxins 7(1):170-186.

Carbon tetrachloride (CC14) induces toxin-mediated liver fibrosis. This toxin is metabolized in the liver by the cytochrome P450 system, which forms the trichloromethyl radical (CCl₃*). This aggressive radical chemically attacks nucleic acid, protein, and lipids, interferes with lipid metabolism and homeostasis, and provokes hepatic steatosis. CC14 has also been shown to induce ER stress. See Borkham-Kamphorst et al., 2020, International Journal of Molecular Sciences 21(15):5230.

Acetaminophen overdose is the leading cause of drug-induced liver failure in the United States. The liver toxicity of acetaminophen is known to be initiated by N-acetylbenzoquinoneimine, an active metabolite produced by the cytochrome P450. Studies have demonstrated a role of ER stress in acetaminophen-induced liver toxicity. See Chen et al., 2014, J Environ Sci Health C Environ Carcinog Ecotoxicol Rev. 32(1): 83-104.

Such stress conditions are capable of inducing the process of cell turnover when present in sufficient amount or intensity and for a sufficient period of time. In certain embodiments, the stress condition that induces cell turnover induces the production of cell turnover factors (e.g. cell turnover factors that promote tissue regeneration) but does not result in cell death. In some embodiments, the stress condition induces cell turnover in a portion of a cell population, e.g., 10%, 20%, 30%, 40%, 50%, 60%, 70%, 80%, 90%, 95% or more cells of the population, such that cell turnover factors (e.g., cell turnover factors that promote tissue regeneration), are produced by the portion of cells in the cell population. Cell death may occur in all or only a fraction of the portion of cells in the cell population.

According to the methods of the invention, the cells are exposed to the stress condition for a sufficient time to induce production of cell turnover factors. In some embodiments, the cell is exposed to the stress condition for at least 1, 2, 3, 4, 5, 6, 7, 8, 9, 10, 20, 30, 40, 50 or 60 minutes. In some embodiments, the cell is exposed to the stress condition for at least 1, 2, 3, 4, 5, 6, 7, 8, 9, 10, 11, 12, 24, 36, 48, 60 or 72 hours.

Any type of cell may be exposed to the stress conditions described herein for the production of cell turnover factors. Suitable cells include, but are not limited to, liver cells, kidney cells, pancreatic cells, muscle cells, bone cells, cells of the intestinal lining, cardiac cells, lung cells, skin cells, neurons, cells of the central nervous system (CNS), epithelial cells, endothelial cells, fibroblasts, and immune cells. In some embodiments, the epithelial cells, endothelial cells, fibroblasts or immune cells are from the liver, kidney, pancreas, muscle, bone, intestinal lining, heart, lung, skin, or the central nervous system. In some embodiments, the liver cells are hepatocytes, hepatic stellate cells, Kupffer cells, or liver simusoidal endothelial cells. In some embodiments, the liver cells comprise one or more of hepatocytes, hepatic stellate cells, Kupffer cells, or liver simusoidal endothelial cells. In some embodiments, the liver cells comprise hepatocytes. In some embodiments, the liver cells are hepatocytes.

In some embodiments, the cells exposed to a stress condition are from the same type of tissue in which the tissue regeneration occurs. For example, in some embodiments, the cells exposed to a stress condition are liver cells, and the tissue in which the tissue regeneration occurs is liver tissue; the cells exposed to a stress condition are kidney cells and the tissue in which the tissue regeneration occurs is kidney tissue; the cells exposed to a stress condition are lung cells and the tissue in which the tissue regeneration occurs is lung tissue; the cells exposed to a stress condition are muscle cells and the tissue in which the tissue regeneration occurs is muscle tissue; the cells exposed to a stress condition are bone cells and the tissue in which the tissue regeneration occurs is bone tissue; the cells exposed to a stress condition are pancreatic cells and the tissue in which the tissue regeneration occurs is pancreatic tissue; the cells exposed to a stress condition are cardiac cells and the tissue in which the tissue regeneration occurs is cardiac tissue; or the cells exposed to a stress condition are cells of the intestinal lining and the tissue in which the tissue regeneration occurs is the lining of the intestine; the cells exposed to a stress condition are skin cells and the tissue in which the tissue regeneration occurs is skin tissue; the cells exposed to a stress condition are cells of the CNS and the tissue in which the tissue regeneration occurs is CNS tissue; the cells exposed to a stress condition are epithelial cells and the tissue in which the tissue regeneration occurs is epithelium; or the cells exposed to a stress condition are endothelial cells and the tissue in which the tissue regeneration occurs is endothelium.

In other embodiments, the cells exposed to a stress condition are not from the same type of tissue in which the tissue regeneration occurs.

In some embodiments, the cells exposed to a stress condition are cancer cells, for example, cells of a sarcoma, melanoma, carcinoma, leukemia, or lymphoma.

The term “sarcoma” generally refers to a tumor which is made up of a substance like the embryonic connective tissue and is generally composed of closely packed cells embedded in a fibrillar or homogeneous substance. Examples of sarcoma cells that may be exposed to a stress condition include, for example, cells from a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, uterine sarcoma, myxoid liposarcoma, leiomyosarcoma, spindle cell sarcoma, desmoplastic sarcoma, and telangiectaltic sarcoma.

The term “melanoma” is taken to mean a tumor arising from the melanocytic system of the skin and other organs. Melanoma cells that can be exposed to a stress condition include, for example, cells from acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinoma cells that may be exposed to a stress condition, as described herein, include, for example, cells from acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, colon adenocarcinoma of colon, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma,

Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, merkel cell carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, cervical squamous cell carcinoma, tonsil squamous cell carcinoma, and carcinoma villosum.

The term “leukemia” refers to a type of cancer of the blood or bone marrow characterized by an abnormal increase of immature white blood cells called “blasts”. Leukemia is a broad term covering a spectrum of diseases. In turn, it is part of the even broader group of diseases affecting the blood, bone marrow, and lymphoid system, which are all known as hematological neoplasms. Leukemias can be divided into four major classifications, acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia. In certain embodiments, leukemias include acute leukemias. In certain embodiments, leukemias include chronic leukemias.

The term “lymphoma” refers to a group of blood cell tumors that develop from lymphatic cells. The two main categories of lymphomas are Hodgkin lymphomas (HL) and non-Hodgkin lymphomas (NHL) Lymphomas include any neoplasms of the lymphatic tissues. The main classes are cancers of the lymphocytes, a type of white blood cell that belongs to both the lymph and the blood and pervades both.

In some embodiments, the cells exposed to a stress condition are from various types of solid tumors, for example breast cancer (e.g. triple negative breast cancer), bladder cancer, genitourinary tract cancer, colon cancer, rectal cancer, endometrial cancer, kidney (renal cell) cancer, pancreatic cancer, prostate cancer, thyroid cancer (e.g. papillary thyroid cancer), skin cancer, bone cancer, brain cancer, cervical cancer, liver cancer, stomach cancer, mouth and oral cancers, esophageal cancer, adenoid cystic cancer, neuroblastoma, testicular cancer, uterine cancer, thyroid cancer, head and neck cancer, kidney cancer, lung cancer (e.g. small cell lung cancer, non-small cell lung cancer), mesothelioma, ovarian cancer, sarcoma, stomach cancer, uterine cancer, cervical cancer, medulloblastoma, and vulvar cancer. In certain embodiments, skin cancer includes melanoma, squamous cell carcinoma, and cutaneous T-cell lymphoma (CTCL).

In one embodiment, the cancer cells are immortalized. In one embodiment, the cancer cells are primary cells isolated from a subject. In some embodiments, the cells exposed to a stress condition do not comprise a cancer cell.

In some embodiments, the cells exposed to a stress condition are immune cells, including but not limited to any one or more of mast cells, Natural Killer (NK) cells, basophils, neutrophils, monocytes, macrophages, dendritic cells, eosinophils, lymphocytes (e.g. B-lymphocytes (B-cells)), and T-lymphocytes (T-cells)). In other embodiments, the cells exposed to a stress condition do not comprise immune cells.

In some embodiments, the cells exposed to a stress condition are blood cells, e.g. erythrocytes, leukocytes (e.g. peripheral blood mononuclear cells (PBMCs)), or thrombocytes. In other embodiments, the cells exposed to a stress condition do not comprise blood cells. In some embodiments, the cells exposed to a stress condition do not comprise leukocytes. In some embodiments, the cells exposed to a stress condition do not comprise peripheral blood mononuclear cells (PBMCs). In some embodiments, the cells exposed to a stress condition do not comprise T-cells. In some embodiments, the cells exposed to a stress condition do not comprise malignant T-cells.

The source of the cells exposed to a stress condition is not limited, and may include cells isolated from the target tissue, organ or subject to which the composition comprising one or more cell turnover factors is administered. For example, in some embodiments, the cells exposed to a stress condition are autologous to the tissue, organ or subject to which the composition comprising one or more cell turnover factors is administered. In some embodiments, the cells exposed to a stress condition are allogeneic to the tissue, organ or subject to which the composition comprising one or more cell turnover factors is administered.

### Compositions Comprising Cell Turnover Factors

According to the methods provided herein, the composition comprising one or more cell turnover factors produced by cells exposed to a stress condition may contain various components in addition to the one or more cell turnover factors, depending, for example, on the method of preparing the composition. For example, in some embodiments, the composition comprises the cells exposed to a stress condition in addition to the one or more cell turnover factors produced by these cells, e.g., the cells comprise the one or more cell turnover factors. In other embodiments, the cells exposed to a stress condition may be separated from the one or more cell turnover factors to prepare the composition. For example, in some embodiments, the composition comprises a cell-free extract prepared from cells exposed to a stress condition, e.g., the cell-free extract comprises the one or more cell turnover factors. Cell-free extracts may be prepared, for example, by centrifuging cells suspended in a culture medium and collecting the supernatant. In one embodiment, the composition comprising one or more cell turnover factors does not comprise the cells that were exposed to the stress condition. In one embodiment, the composition comprising one or more cell turnover factors does not comprise intact cells.

The composition comprising the one or more cell turnover factors may be prepared by culturing cells in a culture medium and exposing the cells to a stress condition as described herein. In one embodiment, conditioned medium containing the one or more cell turnover factors is collected from the cell culture after exposure to the stress condition. The cells may be further cultured after exposure to the stress condition to allow for release of additional cell turnover factors. In some embodiments, the cells are cultured for at least 5, 10, 15, 20, 30, 45 or 60 minutes, or at least 2, 3, 4, 5, 6, 7, 8, 9, 10, 11, 12, 24, 36, 48 or 72 hours after exposure to the stress condition. In one embodiment, the composition comprises conditioned medium from cells exposed to a stress condition. In one embodiment, the one or more cell turnover factors are isolated from the cells exposed to the stress condition, such that the composition comprising one or more cell turnover factors does not contain intact cells.

The composition comprising the one or more cell turnover factors may be fractionated to isolate or concentrate one or more cell turnover factors with tissue regeneration activity. For example, in one embodiment, the composition comprises a functional fraction of the conditioned medium from the cells exposed to the stress condition. In some embodiments, the functional fraction is prepared by treating the conditioned medium with an enzyme (e.g. a protease) to degrade a particular class of compounds in the conditioned medium (e.g. proteins) and increase the relative abundance of other molecules (e.g. small molecules and nucleic acids). Suitable enzymes include, but are not limited to, proteases and nucleases (e.g. RNases or DNases). In some embodiments, the functional fraction comprising the one or more cell turnover factors is resistant to protease digestion or nuclease digestion (e.g. RNAse digestion or DNAse digestion). In some embodiments, the one or more cell turnover factors are resistant to protease digestion or nuclease digestion (e.g. RNAse digestion or DNAse digestion).

Functional fractions of the conditioned medium may also be prepared by isolating molecules based on their molecular weight. For example, in some embodiments, cell turnover factors with a molecular weight of less than 50, 40, 30, 20, 10, 9, 8, 7, 6, 5, 4, 3, 2, 1 or 0.5 are isolated from the conditioned medium. In some embodiments, cell turnover factors with a molecular weight of less than 50, 40, 30, 20, 10, 9, 8, 7, 6, 5, 4, 3, 2, 1 or 0.5 kDa are isolated from the conditioned medium. Any of these values may be used to define a range for the size of the one or more cell turnover factors in the composition. For example, in some embodiments, the one or more cell turnover factors in the composition have a molecular weight of less than 50, 40, 30, 20, 10, 9, 8, 7, 6, 5, 4, 3, 2, 1 or 0.5 kDa. In some embodiments, the one or more cell turnover factors in the composition have a molecular weight of greater than 50, 40, 30, 20, 10, 9, 8, 7, 6, 5, 4, 3, 2, 1 or 0.5 kDa. In some embodiments, the one or more cell turnover factors in the composition have a molecular weight of between 0.5 and 50 kDa, between 0.5 and 25 kDa, between 25 and 50 kDa, between 10 and 20 kDa, between 20 and 30 kDa, between 30 and 40 kDa or between 40 and 50 kDa. Methods for isolating compounds of a particular molecular weight are known in the art. For example, in some embodiments, the conditioned medium is extracted with organic solvent followed by HPLC fractionation. In other embodiments, the conditioned medium is subjected to size exclusion chromatography and different fractions are collected. For example, conditioned medium may be applied to a size exclusion column and fractionated on FPLC.

The functional fractions of the conditioned medium may be evaluated to identify fractions with a particular activity, e.g. tissue regeneration activity. For example, cell proliferation assays such as BrdU and MTT assays could be used to identify fractions of the conditioned medium with tissue regeneration activity.

The BrdU cell Proliferation assay detects 5-bromo-2′-deoxyuridine (BrdU) incorporated into cellular DNA during cell proliferation using an anti-BrdU antibody. When cells are cultured with labeling medium that contains BrdU, this pyrimidine analog is incorporated in place of thymidine into the newly synthesized DNA of proliferating cells. After removing labeling medium, cells are fixed and the DNA is denatured with our fixing/denaturing solution. Then a BrdU mouse mAb is added to detect the incorporated BrdU. The denaturing of DNA is necessary to improve the accessibility of the incorporated BrdU to the detection antibody. Anti-mouse IgG, HRP-linked antibody is then used to recognize the bound detection antibody. HRP substrate TMB is added to develop color. The magnitude of the absorbance for the developed color is proportional to the quantity of BrdU incorporated into cells, which is a direct indication of cell proliferation.

The MTT assay is used to measure cellular metabolic activity as an indicator of cell viability, proliferation and cytotoxicity. This colorimetric assay is based on the reduction of a yellow tetrazolium salt (3-(4,5-dimethylthiazol-2-yl)-2,5-diphenyltetrazolium bromide or MTT) to purple formazan crystals by metabolically active cells. The viable cells contain NAD(P)H-dependent oxidoreductase enzymes which reduce the MTT to formazan. The insoluble formazan crystals are dissolved using a solubilization solution and the resulting colored solution is quantified by measuring absorbance at 500-600 nanometers using a multi-well spectrophotometer. The darker the solution, the greater the number of viable, metabolically active cells.

Additional methods for identifying fractions of the conditioned medium with tissue regeneration activity include, but are not limited to wound healing assays, matrix sprouting, transwell migration assays, endothelial cell tube formation assays, and zymogen assay-matrix degradation. In matrix sprouting, endothelial cells are cultured and then embedded into a collagen matrix and tube formation is analyzed. See Tetzlaff et al., 2018, Bio-Protocol 8(17): e2995. The transwell cell migration assay may be used to measure the effect of a compound or composition on the chemotactic capability of cells toward a chemo-attractant. The effect of the compound or composition on the mode of cell migration and the ability of a cell to invade into a 3-D matrix may also be determined. See Justus et al., 2014, J Vis Exp. (88): 51046.

The skilled artisan will recognize that, in addition to the utility of a functional fraction of the conditioned medium, the functional fraction may be further analyzed to identify, for example, a single cell turnover factor having tissue regeneration activity. In some embodiments, the composition comprises only one cell turnover factor isolated from a cell exposed to a stress condition, and which has tissue regeneration activity. In some embodiments, the composition comprises 2, 3, 4, 5, 6, 7, 8, 9, 10 or more cell turnover factors isolated from a cell exposed to a stress condition, and which have tissue regeneration activity. In some embodiments, the composition comprises at least ten cell turnover factors, e.g. at least ten cell turnover factors that have tissue regeneration activity. In some embodiments, the composition comprises at least two cell turnover factors, e.g. at least two cell turnover factors that have tissue regeneration activity. In some embodiments, the composition comprises only one cell turnover factor, e.g. only one cell turnover factor that has regeneration activity.

In some embodiments, the composition is formulated to target the organ in which tissue regeneration is increased. In some embodiments, the composition is formulated to target liver, kidney, pancreas, muscle, intestinal lining, heart, or lung. In some embodiment, the composition is formulated to target liver. In some embodiments, the composition is formulated to target kidney.

### III. Methods of Increasing Tissue Regeneration

### A. Administration of a Composition Comprising One or More Cell Turnover Factors

The cell turnover factors described herein may be used to increase tissue regeneration in a tissue or organ of a subject, for example, a subject who would benefit from increased tissue regeneration. Accordingly, in some aspects, the disclosure relates to a method of increasing regeneration of a tissue in an organ of a mammalian subject, the method comprising administering to the subject a composition in an amount and for a time sufficient to increase regeneration of the tissue, wherein the composition is made by (a) exposing mammalian cells to a stress condition, and (b) collecting the exposed cells or conditioned media from the exposed cells to create the composition. In some embodiments, the stress condition is thioacetamide.

In some embodiments, the mammalian cells exposed to the stress condition are selected from the group consisting of liver cells, kidney cells, pancreatic cells, muscle cells, bone cells, cells of the intestinal lining, cardiac cells, lung cells, skin cells, neurons, cells of the central nervous system, epithelial cells, endothelial cells, fibroblasts, and immune cells. In some embodiments, the mammalian cells exposed to the stress condition are liver cells (e.g., hepatocytes). In some embodiments, the organ in which tissue regeneration occurs is liver.

In some embodiments, the liver has an injury. In some embodiments, the subject has a liver disorder selected from the group consisting of chronic liver damage, alcoholic steatohepatitis (ASH), drug-induced liver injury, fulminant and late-onset hepatic failure (LOHF), fulminant hepatitis (FH), liver cirrhosis, liver fibrosis, fulminant hepatic failure (FHF), hepatitis B, and hepatitis C. In a particular embodiment, the cells exposed to the stress condition are liver cells (e.g., hepatocytes), and the organ in which tissue regeneration occurs is liver. In a particular embodiment, the stress condition is thioacetamide, the cells exposed to the stress condition are liver cells (e.g., hepatocytes), and the organ in which tissue regeneration occurs is liver.

In certain aspects, the disclosure also relates to a method of delivering a composition comprising one or more cell turnover factors produced by cells exposed to a stress condition to a subject in need of increased tissue regeneration, the method comprising administering the composition to the subject. The disclosure further relates to a method of increasing regeneration of a tissue in an organ of a subject, the method comprising administering to the subject a composition comprising one or more cell turnover factors produced by cells exposed to a stress condition, wherein the composition is administered to the subject in an amount sufficient to increase regeneration of the tissue relative to a subject that is not treated with the composition.

In other embodiments, a tissue or organ is treated in vitro with the composition comprising one or more cell turnover factors produced by cells exposed to a stress condition, and the treated tissue or organ is then transplanted into a subject. For example, in some aspects the disclosure relates to a method of treating a tissue, organ, organoid or organ culture, the method comprising treating a tissue, organ, organoid or organ culture in vitro with a composition comprising one or more cell turnover factors produced by cells exposed to a stress condition. The disclosure also relates to a method of preparing an organ or tissue for transplantation to a subject, the method comprising treating an organ or tissue ex vivo with a composition comprising one or more cell turnover factors produced by cells exposed to a stress condition. The disclosure further relates to a method of increasing tissue regeneration in a transplanted organ or tissue, the method comprising treating an organ or tissue ex vivo with a composition comprising one or more cell turnover factors produced by cells exposed to a stress condition, wherein the organ or tissue is treated with the composition in an amount sufficient to increase tissue regeneration in the organ or tissue after transplantation to a subject relative to an organ or tissue that is not treated with the composition.

Treatment of a tissue or organ in vitro with the composition comprising one or more cell turnover factors produced by cells exposed to a stress condition may also be combined with administration of the composition to a subject as described herein. For example, in some embodiments, the tissue or organ is treated in vitro with the composition comprising one or more cell turnover factors produced by cells exposed to a stress condition, and the tissue or organ is transplanted into a subject. The subject receiving the transplanted tissue or organ may be further administered a composition comprising one or more cell turnover factors produced by cells exposed to a stress condition after transplantation, for example, to further increase tissue regeneration in the transplanted tissue or organ. In some embodiments, the composition comprising one or more cell turnover factors produced by cells exposed to a stress condition used to treat the tissue or organ in vitro is the same composition that is administered to the subject after transplantation. In other embodiments, the composition comprising one or more cell turnover factors produced by cells exposed to a stress condition used to treat the tissue or organ in vitro is different from the composition comprising one or more cell turnover factors produced by cells exposed to a stress condition that is administered to the subject after transplantation.

The composition comprising one or more cell turnover factors may be administered in amounts sufficient to achieve the desired effect on tissue regeneration. For example, in some embodiments, the organ or tissue is treated with the composition in an amount sufficient to increase tissue regeneration in the organ or tissue after transplantation to the subject relative to an organ or tissue that is not treated with the composition. In some embodiments, the organ or tissue is treated with the composition in an amount sufficient to increase survival of the subject after transplantation of the organ or tissue to the subject relative to a subject transplanted with an organ or tissue that is not treated with the composition. In some embodiments, the organ or tissue is treated with the composition in an amount sufficient to improve engraftment of the organ or tissue after transplantation to the subject relative to an organ or tissue that is not treated with the composition. In some embodiments, the organ or tissue is treated with the composition in an amount sufficient to prolong viability of the organ or tissue before transplantation to the subject relative to an organ or tissue that is not treated with the composition.

In some embodiments, the composition is administered to the subject in an amount sufficient to decrease necrosis in the tissue in which the tissue regeneration is increased, relative to a subject that is not treated with the composition. In some embodiments, the composition is administered to the subject in an amount sufficient to decrease steatosis in the tissue in which the tissue regeneration is increased, relative to a subject that is not treated with the composition. In some embodiments, the composition is administered to the subject in an amount sufficient to increase weight of the organ in which the tissue regeneration is increased, relative to a subject that is not treated with the composition. In some embodiment, the composition is administered to the subject in an amount sufficient to increase expression of one or more cell proliferation marker proteins in the tissue, relative to a subject that is not treated with the composition. In some embodiments, the one or more cell proliferation marker proteins are selected from the group consisting of Proliferating Cell Nuclear Antigen (PCNA) and Ki67.

In some embodiments, administration of the composition comprising one or more cell turnover factors increases tissue regeneration, increases survival, improves engraftment, prolongs viability, increases weight of the organ in which tissue regeneration is increased, or increases expression of one or more cell proliferation marker proteins by at least 1%, 2%, 3%, 4%, 5%, 10%, 20%, 30%, 40%, 50%, 60%, 70%, 80%, 90%, 100%, 200%, 300%, 400% or 500% relative to a corresponding control subject that is not administered the composition comprising one or more cell turnover factors. In some embodiments, administration of the composition comprising one or more cell turnover factors decreases necrosis and/or steatosis in the tissue in which the tissue regeneration is increased or by at least 1%, 2%, 3%, 4%, 5%, 10%, 20%, 30%, 40%, 50%, 60%, 70%, 80%, 90% or 100% relative to a corresponding control subject that is not administered the composition comprising one or more cell turnover factors.

In some embodiments, administration of the composition comprising one or more cell turnover factors increases tissue regeneration, increases survival, improves engraftment, prolongs viability, increases weight of the organ in which tissue regeneration is increased, or increases expression of one or more cell proliferation marker proteins in a population of subjects in need of such treatment by at least 1%, 2%, 3%, 4%, 5%, 10%, 20%, 30%, 40%, 50%, 60%, 70%, 80%, 90%, 100%, 200%, 300%, 400% or 500% relative to a corresponding population of control subjects that is not administered the composition comprising one or more cell turnover factors. In some embodiments, administration of the composition comprising one or more cell turnover factors decreases necrosis and/or steatosis in the tissue in which the tissue regeneration is increased in a population of subjects in need of such treatment by at least 1%, 2%, 3%, 4%, 5%, 10%, 20%, 30%, 40%, 50%, 60%, 70%, 80%, 90% or 100% relative to a corresponding population of control subjects that is not administered the composition comprising one or more cell turnover factors.

In some embodiments, tissue regeneration in an organ (e.g. liver, lung, pancreas, kidney, heart, muscle, skin and organs of the gastrointestinal tract (e.g. mouth, esophagus, stomach, small intestine, large intestine, and anus)) is determined by measuring organ function before and after administration of the composition comprising one or more cell turnover factors. Exemplary methods of measuring organ function as a means of determining tissue regeneration are described below.

Liver

Liver function may be measured in a subject by determining the level of one or more compounds (e.g. proteins) associated with liver function in a sample obtained from the subject. For example, the level of one or more compounds associated with liver function may be determined before and after administration of a composition comprising one or more cell turnover factors to measure the effect of the composition on liver function. Suitable samples include, but are not limited to a solid tissue (e.g. a liver tissue), cells (e.g. liver cells), whole blood, serum, plasma, saliva, urine and stool. Suitable compounds associated with liver function include, but are not limited to, Alanine transaminase (ALT), Aspartate transaminase (AST), Alkaline phosphatase (ALP), albumin, bilirubin, Gamma-glutamyltransferase (GGT), and L-lactate dehydrogenase (LD).

In some embodiments, elevated levels of the compound associated with liver function are indicative of liver damage, such that a decrease in levels of the compound after treatment with the composition comprising one or more cell turnover factors is indicative of improved liver function and liver tissue regeneration. For example, ALT is an enzyme found in the liver that helps convert proteins into energy for liver cells. When the liver is damaged, ALT is released into the bloodstream and levels increase. AST is an enzyme that helps metabolize amino acids. Like ALT, AST is normally present in blood at low levels. An increase in AST levels may indicate liver damage, disease or muscle damage. ALP is an enzyme found in the liver and bone and is important for breaking down proteins. Higher-than-normal levels of ALP may indicate liver damage or disease, such as a blocked bile duct, or certain bone diseases. Bilirubin is a substance produced during the normal breakdown of red blood cells. Bilirubin passes through the liver and is excreted in stool. Elevated levels of bilirubin (jaundice) might indicate liver damage or disease or certain types of anemia. GGT is an enzyme in the blood. Higher-than-normal levels may indicate liver or bile duct damage. LD is an enzyme found in the liver. Elevated levels of LD may indicate liver damage. Because elevated levels of ALT, AST, ALP, bilirubin, GGT and LD are indicative of liver damage, a decrease in levels of these compounds after treatment with the composition comprising one or more cell turnover factors is indicative of improved liver function and liver tissue regeneration.

In some embodiments, a reduced level of the compound associated with liver function is indicative of liver damage, such that an increase in levels of the compound after treatment with the composition comprising one or more cell turnover factors in indicative of improved liver function and liver tissue regeneration. For example, albumin is one of several proteins made in the liver that is needed to fight infections and to perform other functions. Lower-than-normal levels of albumin may indicate liver damage or disease. Accordingly, increased levels of albumin after administration of a composition comprising one or more cell turnover factors is indicative of improved liver function and liver tissue regeneration.

Liver function may also be measured by determining total protein levels in the blood or prothrombin time (PT). Reduced total protein levels in the blood may be indicative of reduced liver function. Accordingly, increased levels of total protein after administration of a composition comprising one or more cell turnover factors is indicative of improved liver function and liver tissue regeneration.

Prothrombin time (PT) may also be used to measure liver function. PT is the time it takes blood to clot. Increased PT may indicate liver damage, but may also be elevated by certain blood-thinning drugs, such as warfarin. Accordingly, decreased PT after administration of a composition comprising one or more cell turnover factors is indicative of improved liver function and liver tissue regeneration.

Lung

Methods of measuring lung function as a means of detecting lung tissue regeneration in a subject include, but are not limited to, spirometry, lung volume tests, lung diffusion capacity tests, pulse oximetry, arterial blood gas tests and fractional exhaled nitric oxide tests. One or more of these tests may be conducted before and after administering a composition comprising one or more cell turnover factors for detecting lung tissue regeneration.

Spirometry measures the rate of air flow and estimates lung size. For this test, you will breathe multiple times, with regular and maximal effort, through a tube that is connected to a computer. Some subjects may feel lightheaded or tired from the required breathing effort.

Lung volume tests are the most accurate way to measure how much air the lungs can hold. The procedure is similar to spirometry, except that the subject will be in a small room with clear walls. Some subjects may feel lightheaded or tired from the required breathing effort.

Lung diffusion capacity assesses how well oxygen gets into the blood from the air that is breathed. For this test, the subject breathes in and out through a tube for several minutes without having to breathe intensely. The subject may also have blood drawn to measure the level of hemoglobin in the blood.

Pulse oximetry estimates oxygen levels in the blood. For this test, a probe is placed on a finger or another skin surface such as an ear. It causes no pain and has few or no risks.

Arterial blood gas tests directly measure the levels of gases, such as oxygen and carbon dioxide, in the blood. Arterial blood gas tests are usually performed in a hospital, but may be done in a doctor's office. For this test, blood is taken from an artery, usually in the wrist where the pulse is measured, and levels of gas in the blood are determined.

Fractional exhaled nitric oxide tests measure how much nitric oxide is in the air that is exhaled. For this test, the subject breathes out into a tube that is connected to a portable device that measures nitric oxide levels. This test requires steady but not intense breathing. It has few or no risks.

Pancreas

Methods of measuring pancreas function as a means of detecting pancreas tissue regeneration in a subject include, but are not limited to, secretin pancreatic function test, and fecal elastase test. Regeneration of pancreatic tissue may also be detected by imaging methods including, but not limited to computed tomography (CT) scan with contrast dye, abdominal ultrasound, endoscopic retrograde cholangiopancreatography (ERCP), endoscopic ultrasound, and magnetic resonance cholangiopancreatography. One or more of these tests may be conducted before and after administering a composition comprising one or more cell turnover factors for detecting pancreatic tissue regeneration.

The secretin pancreatic function test measures the ability of the pancreas to respond to the hormone secretin. The small intestine produces secretin in the presence of partially digested food. Normally, secretin stimulates the pancreas to secrete a fluid with a high concentration of bicarbonate. This fluid neutralizes stomach acid and is necessary to allow a number of enzymes to function in the breakdown and absorption of food. People with diseases involving the pancreas (for example, chronic pancreatitis, cystic fibrosis, or pancreatic cancer) might have abnormal pancreatic function. In performing a secretin pancreatic function test, a healthcare professional places a tube down the throat, into the stomach, then into the duodenum (upper section of small intestine). Secretin is inserted and the contents of the duodenal secretions are aspirated (removed with suction) for about an hour and analyzed.

The fecal elastase test measures elastase, an enzyme found in fluids produced by the pancreas. Elastase digests and degrades various kinds of proteins. During this test, a patient's stool sample is analyzed for the presence of elastase.

The computed tomography (CT) scan with contrast dye helps to rule out other causes of abdominal pain and can also determine whether there is inflammation (swelling), scarring, or fluid collections in or around the pancreas.

An abdominal ultrasound can detect gallstones and fluid from inflammation in the abdomen (ascites). It also can show an enlarged common bile duct, an abscess, or a pseudocyst.

During an endoscopic retrograde cholangiopancreatography (ERCP), a healthcare professional places a tube down the throat, into the stomach, then into the small intestine. A small catheter is passed into the pancreas and bile ducts, and dye is injected to help the doctor see the structure of the common bile duct, other bile ducts, and the pancreatic duct on an X-ray.

During an endoscopic ultrasound, a probe attached to a lighted scope is placed down the throat and into the stomach. Sound waves show images of organs in the abdomen. Endoscopic ultrasound might reveal gallstones and can be helpful in diagnosing severe pancreatitis when an invasive test such as ERCP might make the condition worse.

Magnetic resonance cholangiopancreatography is a type of magnetic resonance imaging (MRI) that can be used to look at the bile ducts and the pancreatic duct. MRI/MRCP gives very good imaging of the pancreas and does not use radiation.

Kidney

Methods of measuring kidney function as a means of detecting kidney tissue regeneration in a subject include blood tests such as serum creatinine, glomerular filtration rate (GFR), blood urea nitrogen (BUN); and imaging tests such as ultrasound and CT scan.

Creatinine is a waste product that comes from the normal wear and tear on muscles of the body. Creatinine levels in the blood can vary depending on age, race and body size. A creatinine level of greater than 1.2 for women and greater than 1.4 for men may be an early sign that the kidneys are not working properly. As kidney disease progresses, the level of creatinine in the blood rises.

The glomerular filtration rate (GFR) test (calculated by mathematical formula using the Modification of Diet in Renal Disease (MDRD) or the Chronic Kidney Disease Epidemiology Collaboration (CKD-EPI) equation) measures how well the kidneys are removing wastes and excess fluid from the blood. It is calculated from the serum creatinine level using age and gender with adjustment for those of African American descent. Normal GFR can vary according to age (as a subject ages it can decrease). The normal value for GFR is 90 or above. A GFR below 60 is a sign that the kidneys are not working properly. Once the GFR decreases below 15, one is at high risk for needing treatment for kidney failure, such as dialysis or a kidney transplant.

Blood Urea Nitrogen (BUN). Urea nitrogen comes from the breakdown of protein in foods. A normal BUN level is between 7 and 20. As kidney function decreases, the BUN level rises.

Ultrasound uses sound waves to image the kidney. It may be used to look for abnormalities in size or position of the kidneys or for obstructions such as stones or tumors.

A CT Scan uses X-rays to image the kidneys. It may also be used to look for structural abnormalities and the presence of obstructions. This test may require the use of intravenous contrast dye which can be of concern for those with kidney disease.

Kidney tissue may also be evaluated through a kidney biopsy, and/or by urine tests such as urinalysis, urine protein tests, microalbuminuria, and creatinine clearance.

A biopsy may be done occasionally for one or more of the following reasons: to identify a specific disease process and determine whether it will respond to treatment; to evaluate the amount of damage that has occurred in the kidney; and/or to find out why a kidney transplant may not be doing well. A kidney biopsy is performed by using a thin needle with a sharp cutting edge to slice small pieces of kidney tissue for examination under a microscope.

Some urine tests require only a few tablespoons of urine. Other tests require collection of all urine produced for a full 24 hours. A 24-hour urine test shows how much urine the kidney produces, can give a more accurate measurement of how well the kidney is working and how much protein leaks from the kidney into the urine in one day.

Urinalysis includes microscopic examination of a urine sample as well as a dipstick test. The dipstick is a chemically treated strip, which is dipped into a urine sample. The strip changes color in the presence of abnormalities such as excess amounts of protein, blood, pus, bacteria and sugar. A urinalysis can help to detect a variety of kidney and urinary tract disorders, including chronic kidney disease, diabetes, bladder infections and kidney stones.

A urine protein test may be done as part of a urinalysis or by a separate dipstick test. An excess amount of protein in the urine is called proteinuria. A positive dipstick test (1+ or greater) should be confirmed using a more specific dipstick test such as an albumin specific dipstick or a quantitative measurement such as an albumin-to-creatinine ratio.

Microalbuminuria is a more sensitive dipstick test which can detect a tiny amount of protein called albumin in the urine. People who have an increased risk of developing kidney disease, such as those with diabetes or high blood pressure, should have this test or an albumin-to-creatinine ratio if their standard dipstick test for proteinuria is negative.

Creatinine is a waste product that comes from the normal wear and tear on muscles of the body. A creatinine clearance test compares the creatinine in a 24-hour sample of urine to the creatinine level in your blood to show how much waste products the kidneys are filtering out each minute.

Heart

Methods of measuring heart function as a means of detecting heart tissue regeneration in a subject include, but are not limited to, an echocardiogram, transesophageal echocardiography (TEE), an electrocardiogram (ECG or EKG), magnetic resonance imaging (MRI), a CT scan, an exercise cardiac stress test, a pharmacologic stress test, a tilt test, an ambulatory rhythm monitoring tests, Holter monitoring, mobile cardiac telemetry (MCT), and coronary angiogram.

An echocardiogram uses sound waves to produce images of the heart. This common test allows a physician to see how the heart is beating and how blood is moving through the heart. Images from an echocardiogram are used to identify various abnormalities in the heart muscle and valves. This test can be done at rest or with exercise to elevate the heart rate (see exercise cardiac stress test below).

Transesophageal echocardiography (TEE) uses high-frequency sound waves (ultrasound) to make detailed pictures of the heart and the arteries that lead to and from it.

The echo transducer that produces the sound waves for TEE is attached to a thin tube that passes through your mouth, down your throat and into your esophagus, which is very close to the upper chambers of the heart.

An electrocardiogram (ECG or EKG) measures the electrical activity of the heartbeat to provide two kinds of information. First, by measuring time intervals on the ECG, a doctor can determine how long the electrical wave takes to pass through the heart. Finding out how long a wave takes to travel from one part of the heart to the next shows if the electrical activity is normal, slow, fast or irregular. Second, by measuring the amount of electrical activity passing through the heart muscle, a cardiologist may be able to find out if parts of the heart are too large or overworked.

Magnetic resonance imaging (MRI) uses a magnetic field and radiofrequency waves to create detailed pictures of organs and structures inside the body. It can be used to examine the heart and blood vessels and to identify areas of the brain affected by stroke.

A CT scan is an X-ray imaging technique that uses a computer to produce cross-sectional images of the heart. Also referred to as cardiac computed tomography, computerized axial tomography or CAT scan, it can be used to examine the heart and blood vessels for problems. It's also used to identify whether blood vessels in the brain have been affected by stroke.

An exercise cardiac stress test, also called an exercise tolerance test (ETT), shows whether the heart's blood supply is sufficient and if the heart rhythm is normal during exercise on a treadmill or stationary bicycle. The test monitors the level of tiredness, heart rate, breathing, blood pressure and heart activity while exercising. This test may be done in combination with nuclear imaging or echocardiography.

Pharmacologic stress test: Medication is given through an IV line in the arm to dilate the arteries, which increases the heart rate and blood flow, similar to the effects of exercise. This test may be done in combination with nuclear imaging, echocardiography or MRI.

Tilt test: Often used to determine why a patient feels faint or lightheaded. During the test, the patient lies on a table that is slowly tilted upward. The test measures how blood pressure and heart rate respond to the force of gravity. A nurse or technician keeps track of blood pressure and heart rate (pulse) to see how they change during the test.

Ambulatory rhythm monitoring tests: Holter monitoring, event recorders and mobile cardiac telemetry (MCT) are ambulatory monitoring tests done to study your heart rhythm for a prolonged period of time on an outpatient basis.

Coronary angiogram: A type of X-ray used to examine the coronary arteries supplying blood to the heart. A catheter is inserted into a blood vessel in your arm or groin and fed up to your heart and coronary arteries. Special dye is then injected through the catheter and images are taken.

Muscle

Methods of measuring muscle function as a means of detecting muscle tissue regeneration in a subject include, but are not limited to, tests of grip strength, strength of upper limbs and lower limbs, maximal strength, leg press and knee extension, gait speed test, chair stand test (CST), short physical performance battery (SPPB), and the timed-get-up-and-go (TUG) test. These tests are described, for example, in Beaudart, et al. 2019, Calcif Tissue Int 105, 1-14.

Maximal strength is quantified through the 1RM (1 repetition maximum resistance), wherein the evaluation is carried out at the highest resistance for which the subject can complete the exercise once. To find the 1RM, the exercise is repeated several times at increasing resistance until failure to complete a single repetition.

Gait Speed Test—Two main types of gait speed tests exist; the short-distance walk tests (2.4-m distance, 4-m distance, 6-m distance and 10-m distance) and the long-distance walk tests (400-m walk test and 6-min walk test).

Gastrointestinal (GI) Tract

The upper GI tract is generally considered to be the mouth, esophagus, stomach, and the first part of the small intestine (duodenum). The lower GI tract runs from the small intestine to the large intestine (colon) to the anus. Methods of measuring upper GI tract function as a means of detecting upper GI tissue regeneration in a subject include, but are not limited to, an upper GI Series (barium swallow or barium meal), gastroscopy, Endoscopic Retrograde Cholangiopancreatography (ERCP), Endoscopic Ultrasound, pH Monitoring, and Esophageal/Gastric Manometry. Method of measuring lower GI tract function as a means of detecting lower GI tissue regeneration include colonoscopy, barium enema, flexible sigmoidoscopy, virtual colonoscopy, capsule endoscopy, and anorectal manometry. Additional methods of measuring GI tract function as a means of detecting GI tissue regeneration in a subject include, but are not limited to, fecal calprotectin test, fecal occult blood test, fecal immunochemical test (FIT), Hydrogen Breath Test, Lactose Tolerance Test, Stool Acidity Test, Liver Biopsy, FibroScan®, Magnetic Resonance Imaging and Ultrasound.

Skin

Skin tissue regeneration may be determined through visual evaluation, for example by determining a keloid score. Keloids are raised overgrowths of scar tissue that occur at the site of a skin injury. Keloid score may be determined, for example, by the Patient and Observer Scar Assessment Scale (POSAS). See Draaijers et al., 2004, Plast Reconstr Surg 113:1960-1965. The scar is rated numerically on a ten-step scale by both the patient and doctor on six items: vascularity, pigmentation, thickness, relief, pliability, and surface area on the Observer Scale. The Patient Scale consists of pain, itchiness, color, stiffness, thickness, and irregularity of the scar.

In some embodiments, the organ treated with the compositions described herein is a solid organ. The term “solid organ” as used herein refers to an internal organ that has a firm tissue consistency and is neither hollow (such as the organs of the gastrointestinal tract) nor liquid (such as blood). Solid organs include, but are not limited to liver, kidney, pancreas, muscle, heart and lung. In some embodiments, the organ is an organ of the gastrointestinal tract. Organs of the gastrointestinal tract include the esophagus, stomach, small intestine and large intestine.

In some embodiments, the organ treated with the compositions described herein has an injury, for example, an injury caused by a drug, a toxin, viral infection, or surgery to the organ. The composition may be administered to the subject after the organ injury, for example to improve tissue regeneration in the organ and/or improve survival of the subject.

In some embodiments, the composition comprising one or more cell turnover factors is administered to a subject is in need of surgery to an organ. The composition may be administered to the subject before surgery to the organ and/or after surgery to the organ. The surgery may comprise cancer resection involving the organ.

Any tissue capable of regeneration may be treated with the compositions described herein. In some embodiments, the tissue is selected from the group consisting of liver tissue, kidney tissue, pancreas tissue, muscle tissue, intestinal lining, cardiac tissue and lung tissue.

The subject may have a particular disorder associated with the tissue or organ that is to be regenerated. For example, in some embodiments, the tissue to be regenerated is liver tissue and the subject has a disorder selected from the group consisting of chronic liver damage, alcoholic steatohepatitis (ASH), drug-induced liver injury, fulminant and late-onset hepatic failure (LOHF), fulminant hepatitis (FH), liver cirrhosis, liver fibrosis, fulminant hepatic failure (FHF), hepatitis B, and hepatitis C.

In some embodiments, the tissue to be regenerated is kidney tissue and the subject has a disorder or condition selected from the group consisting of diabetes mellitus, rheumatoid arthritis, nephritic syndrome, nephrotic syndrome, hypertension nephropathy, polycystic kidney disease, progressive chronic kidney disease, chronic renal failure, Fabry disease, cystinosis, nephronophthisis, Alport's syndrome, reperfusion injury, acute kidney injury, kidney fibrosis and a kidney transplant.

In some embodiments, the tissue to be regenerated is pancreatic tissue and the subject has a disorder selected from the group consisting of pancreatic cancer, diabetes mellitus, insulin resistance, hypoglycemia, hyperglycemia, lipase deficiency, cholecystokinin (CCK) deficiency, acute pancreatitis, chronic pancreatitis and hereditary pancreatitis.

In some embodiments, the tissue to be regenerated is intestinal lining tissue and the subject has a disorder or condition selected from the group consisting of an inflammatory gastrointestinal disorder, Crohn's disease, inflammatory bowel disease (IBD), diverticulitis, parasitic infection, and bacterial infection, a functional gastrointestinal disorder, ulcerative colitis (UC), and a surgical resection of the intestines.

In some embodiments, the tissue to be regenerated is cardiac tissue and the subject has a cardiovascular disease. Cardiovascular diseases that may be treated by the methods described herein include, but are not limited to, myocardial infarction, heart failure, heart injury by ischemic event, and heart injury by a non-ischemic event.

In some embodiments, the tissue to be regenerated is lung tissue and the subject has a disorder or condition selected from the group consisting of acute respiratory distress syndrome (ARDS), chronic obstructive pulmonary disease (COPD), lung cancer, bronchiolitis obliterans organizing pneumonia (BOOP), Coronavirus Disease 2019 (COVID-19), mesothelioma, and cystic fibrosis, asthma, idiopathic pulmonary fibrosis, lung failure due to aging, pulmonary fibrosis, interstitial lung disease (ILD), pulmonary arterial hypertension and al-antitrypsin disorder.

In some embodiments, the tissue to be regenerated is muscle tissue and the subject has a disorder or condition selected from the group consisting of myositis, autoimmune disease, amyotrophic lateral sclerosis (ALS), sarcopenia, pediatric Charcot-Marie-Tooth disease, muscle loss due to aging, muscle strain from injury, muscle atrophy, muscular dystrophy, dermatomyositis, Guillain-Barré syndrome, multiple sclerosis, polio and polymyositis. In some embodiments, the injury is a sports injury. In some embodiments, the muscle atrophy is spinal muscular atrophy (SMA). In some embodiments, the muscular dystrophy is selected from the group consisting of Duchenne muscular dystrophy, Becker muscular dystrophy, congenital muscular dystrophy, myotonic dystrophy (also known as Steinert's disease or dystrophia myotonica), facioscapulohumeral muscular dystrophy (FSHD), limb-girdle muscular dystrophy, oculopharyngeal muscular dystrophy (OPMD), distal muscular dystrophy (also known as distal myopathy), and Emery-Dreifuss muscular dystrophy.

The methods described herein may further comprise a step of preparing the composition comprising the one or more cell turnover factors. In some embodiments, the step of preparing the composition comprises exposing the cells to the stress condition.

B. Administration of a Compound that Induces Cell Turnover

The disclosure also relates to the use of compounds that induce cell turnover to increase tissue generation by directly administering the compound to a subject, organ or tissue. Induction of cell turnover by the compound results in the production of cell turnover factors in a target cell of the tissue or subject that promote tissue regeneration in the subject, organ or tissue. For example, in certain aspects, the disclosure relates to a method of increasing regeneration of a tissue in an organ of a subject, the method comprising administering to the subject a composition comprising a compound that induces cell turnover of a target cell in the subject, wherein the composition is administered in an amount sufficient to increase regeneration of the tissue relative to a subject that is not treated with the composition.

Any of the compounds that induce cell turnover described herein (e.g. small molecules, nucleic acids, and proteins that induce cell turnover) may be administered to a tissue, organ or subject to increase regeneration of tissue. In some embodiments, the compound administered to a tissue or subject to increase regeneration of tissue is a toxin. For example, a toxin may be administered to a subject or tissue at a dose that is sufficient to induce cell turnover and the production of cell turnover factors, but is lower than the dose that causes severe damage to the tissue.

In some embodiments, the compound administered to a tissue or subject to increase regeneration of tissue induces ER stress and/or an unfolded protein response. Compounds that induce ER stress and/or an unfolded protein response are described herein and include, but are not limited to thioacetamide, tunicamycin, PhenolaTi, Zearalenone, Shiga toxin-2, carbon tetrachloride (CCL4) and acetaminophen. In some embodiments, the compound administered to a tissue or subject to increase regeneration of tissue is an anti-neoplastic agent, for example, one of the anti-neoplastic agents described herein. In some embodiments, the compound administered to a tissue or subject to increase regeneration of tissue is not a compound that induces iron-dependent cell turnover, e.g. ferroptosis.

The compound that induces cell turnover may be targeted to a particular cell, tissue or organ to induce production of cell turnover factors by a target cell that promote tissue regeneration. Various methods known in the art may be used for targeting the compound to a target cell, tissue or organ. For example, in some embodiments, the compound that induces cell turnover is targeted to a particular cell, tissue or organ by conjugation to an antibody that is specific to the target cell, tissue or organ. For example, in some embodiments, the compound that induces cell turnover is an antibody-drug conjugate. The antibody may be targeted to the tissue or organ in which tissue regeneration is increased. In some embodiments, the antibody specifically binds to a tissue-specific antigen of the organ in which tissue regeneration is increased. In some embodiments, the antibody is targeted to the liver. In some embodiments, the antibody is targeted to the kidney.

In some embodiments, the compound (e.g. a toxin) is targeted to a particular organ or tissue through the use of a particular formulation or encapsulation method. For example, in some embodiments, the formulation or encapsulation method targets the compound to the liver. In some embodiments, the formulation or encapsulation method targets the compound to the kidney. Examples of formulations and encapsulation that target compounds to the liver or kidney are provided in Table 1 below.

TABLE 1
Formulations and encapsulation methods that target to the liver or kidney

| | Tissue | |
|---|---|---|
| Method | targeted | Reference |
| label carriers (polymers, human serum albumin, and | liver | 3 |
| recombinant high density lipoproteins) with ASGP-R-specific | | |
| ligands (galactose, lactose, acetylgalactosamine, asialofetuin) | | |
| linear aminopolysaccharide chitosan and its derivatives | liver | 3 |
| Lactose conjugated to polyion complex micelles consisting of | liver | 3 |
| polyethylene glycol grafted to chitosan | | |
| modification of dextran with galactose and mannose | liver | 3 |
| polymers functionalized with galactosamine | liver | 3 |
| HepDirect prodrug | liver | 3 |
| Covalent attachment to bile acids | liver | 3 |
| Poly(vinylbenzyl-O--D-galactopyranosyl-D-gluconamide) | liver | 3 |
| (PVLA) nanoparticles | | |
| apolipoproteins | Liver | 1 |
| Mannose/fucose-modified modified liposomes | Liver | 1 |
| Coupling to ligands of the hyaluran receptor | Liver | 1 |
| Coupling to entities that bind the mannose 6- | Liver | 1 |
| phosphate/insulin-like growth factor II receptor, the collagen | | |
| type VI receptor, and the platelet derived growth factor β | | |
| (PDGF-β) receptor | | |
| liposomes substituted with the collagen type VI receptor | Liver | 1 |
| recognizing peptide | | |
| Exosomes displaying targeting peptides, proteins, Fc domains | Liver, kidney | 7 |
| Conjugates of drug with glycosylated | Liver | 2 |
| poly-L-lysine | | |
| arabinogalactan or glycosylated lipoprotein carriers | Liver | 2 |
| Dextran modification with galactose and mannose | Liver | 2 |
| Envelope HBV proteins (e.g., recombinant large protein, L- | Liver | 2 |
| protein) that form hollow virus-like nanoparticles (~80 nm), | | |
| are so-called bionanocapsules (BNC), | | |
| solid lipid nanoparticles (SLN) | Liver | 2 |
| nosiheptide-loaded liposomes | Liver | 2 |
| amino-acid prodrugs that, after delivery, require activation by | Kidney | 6 |
| kidney-selective enzymes, such as such as L-amino acid | | |
| decarboxylase, β-lyase and N-acetyl transferase, or γ- | | |
| glutamyl transpeptidase (GGT) | | |
| Drugs conjugated to LMWPs, such as lysozyme, cytochrome-c | Kidney | 6 |
| and aprotinin | | |
| Polyethylene glycol (PEG)-grafted liposomes | Kidney | 5 |
| (PEG-liposomes) with a diameter of around 100 nm | | |
| 3,5-dipentadecyloxybenzenecarboxamidine hydrochloride | Kidney | 6 |
| modified liposomes | | |
| lipid-based nanocarriers called SAINT-O-Somes, which were | Kidney | 6 |
| conjugated with the anti-VCAM-1 antibody | | |
| ultrasmall colloidal nanocarriers with brushlike PEG corona | Kidney | 6 |
| and hydrophobic polycaprolactone core. The particle size | | |
| range was tuned down to 5 to 30 nm to promote GFB | | |
| permeation and podocyte accumulation | | |
| Water soluble polymers such as N-vinylpyrrolidone | Kidney | 6 |
| copolymerized with an anionic monomer | | |
| N-Acetylated low molecular weight chitosan | Kidney | 6 |
| Folic acid based prodrugs | Kidney | 6 |
| hyaluronic acid (HA based prodrugs | Kidney | 6 |
| PEG conjugated with poly(lactic-co-glycolic acid) prepared | Kidney | 6 |
| into PLGA-PEG MNPs with a diameter of 400 nm | | |

1. Poelstra K, Prakash J, Beljaars L. Drug targeting to the diseased liver. J Control Release. 2012;161(2):188-197. doi:10.1016/j.jconrel.2012.02.011

2. Cuestas M L, Mathet V L, Oubiña J R, Sosnik A. Drug delivery systems and liver targeting for the improved pharmacotherapy of the hepatitis B virus (HBV) infection. Pharm Res. 2010;27(7):1184-1202. doi:10.1007/s11095-010-0112-z

3. Singh L, Indermun S, Govender M, et al. Drug Delivery Strategies for Antivirals against Hepatitis B Virus. Viruses. 2018;10(5):267. Published 2018 May 17. doi:10.3390/v10050267

5. Liu C P, Hu Y, Lin J C, Fu H L, Lim L Y, Yuan Z X. Targeting strategies for drug delivery to the kidney: From renal glomeruli to tubules. Med Res Rev. 2019;39(2):561-578. doi:10.1002/med.21532

6. Haas M, Moolenaar F, Meijer D K, de Zeeuw D. Specific drug delivery to the kidney. Cardiovasc Drugs Ther. 2002;16(6):489-496. doi:10.1023/a:1022913709849

7. Barile L, Vassalli G. Exosomes: Therapy delivery tools and biomarkers of diseases. Pharmacol Ther. 2017;174:63-78. doi:10.1016/j.pharmthera.2017.02.020

In some embodiments, the compound that induces cell turnover is targeted to a cell in the organ in which tissue regeneration is increased. For example, in some embodiments, the target cell is a liver cell and the tissue that is regenerated is liver tissue. In some embodiments, the target cell is a kidney cell and the tissue that is regenerated is kidney tissue. In some embodiments, the target cell is a lung cell and the tissue that is regenerated is lung tissue; the target cell is a muscle cell and the tissue that is regenerated is muscle tissue; the target cell is a bone cell and the tissue that is regenerated is bone tissue; the target cell is a pancreatic cell and the tissue that is regenerated is pancreatic tissue; the target cell is a cardiac cell and the tissue that is regenerated is cardiac tissue; the target cell is a cell of the intestinal lining and the tissue that is regenerated is the lining of the intestine; the target cell is a skin cell and the tissue that is regenerated is skin tissue; the target cell is a cell of the CNS and the tissue that is regenerated is CNS tissue; the target cell is an epithelial cell and the tissue that is regenerated is epithelium; or the target cell is an endothelial cell and the tissue that is regenerated is endothelium.

The target cell may also be in a tissue adjacent to the organ or tissue in which tissue regeneration is increased. For example, in some embodiments, the target cell is within 50 μm, 100 μm, 500 μm, 1 mm, or 2 mm of the organ or tissue in which tissue regeneration is increased.

In some embodiments, the target cell for the compound that induces cell turnover is resident within the tissue that is regenerated, but is different from the type of cell that is regenerated. For example, in some embodiments the target cell is an epithelial cell, a fibroblast, an endothelial cell or an immune cell that is resident in the liver, and cell turnover factors produced by the target cell induce regeneration of liver tissue. In some embodiments the target cell is an epithelial cell, a fibroblast, an endothelial cell or an immune cell that is resident in the kidney, and cell turnover factors produced by the target cell induce regeneration of kidney tissue. In some embodiments the target cell is an epithelial cell, a fibroblast, an endothelial cell or an immune cell that is resident in the lung, and cell turnover factors produced by the target cell induce regeneration of lung tissue; the target cell is an epithelial cell, a fibroblast, an endothelial cell or an immune cell that is resident in bone, and cell turnover factors produced by the target cell induce regeneration of bone tissue; the target cell is an epithelial cell, a fibroblast, an endothelial cell or an immune cell that is resident in muscle, and cell turnover factors produced by the target cell induce regeneration of muscle tissue; the target cell is an epithelial cell, a fibroblast, an endothelial cell or an immune cell that is resident in the pancreas, and cell turnover factors produced by the target cell induce regeneration of pancreatic tissue, the target cell is an epithelial cell, a fibroblast, an endothelial cell or an immune cell that is resident in the heart, and cell turnover factors produced by the target cell induce regeneration of cardiac tissue; the target cell is an epithelial cell, a fibroblast, an endothelial cell or an immune cell that is resident in the intestinal lining, and cell turnover factors produced by the target cell induce regeneration of intestinal lining tissue; or the target cell is an epithelial cell, a fibroblast, an endothelial cell or an immune cell that is resident in the CNS, and cell turnover factors produced by the target cell induce regeneration of CNS tissue. In some embodiments, the immune cell is a monocyte or a macrophage.

### V. Pharmaceutical Compositions and Modes of Administration

The pharmaceutical compositions described herein may be administered to a subject in any suitable formulation. These include, for example, liquid, semi-solid, and solid dosage forms, The preferred form depends on the intended mode of administration and therapeutic application.

In certain embodiments the composition is suitable for oral administration. In certain embodiments, the formulation is suitable for parenteral administration, including topical administration and intravenous, intraperitoneal, intramuscular, and subcutaneous, injections. In a particular embodiment, the composition is suitable for intravenous administration.

Pharmaceutical compositions for parenteral administration include aqueous solutions of the active compounds in water-soluble form. For intravenous administration, the formulation may be an aqueous solution. The aqueous solution may include Hank's solution, Ringer's solution, phosphate buffered saline (PBS), physiological saline buffer or other suitable salts or combinations to achieve the appropriate pH and osmolarity for parenterally delivered formulations. Aqueous solutions can be used to dilute the formulations for administration to the desired concentration. The aqueous solution may contain substances which increase the viscosity of the solution, such as sodium carboxymethyl cellulose, sorbitol, or dextran. In some embodiments, the formulation includes a phosphate buffer saline solution which contains sodium phosphate dibasic, potassium phosphate monobasic, potassium chloride, sodium chloride and water for injection.

Formulations suitable for topical administration include liquid or semi-liquid preparations suitable for penetration through the skin, such as liniments, lotions, creams, ointments or pastes, and drops suitable for administration to the eye, ear, or nose. Formulations suitable for oral administration include preparations containing an inert diluent or an assimilable edible carrier. The formulation for oral administration may be enclosed in hard or soft shell gelatin capsule, or it may be compressed into tablets, or it may be incorporated directly with the food of the diet. When the dosage unit form is a capsule, it may contain, in addition to materials of the above type, a liquid carrier. Various other materials may be present as coatings or to otherwise modify the physical form of the dosage unit. Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically.

As will be readily apparent to one skilled in the art, the useful in vivo dosage to be administered and the particular mode of administration will vary depending upon the age, body weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, animal models, and in vitro studies.

In certain embodiments, the composition is delivered orally. In certain embodiments, the composition is administered parenterally. In certain embodiments, the compositions is delivered by injection or infusion. In certain embodiments, the composition is delivered topically including transmucosally. In certain embodiments, the composition is delivered by inhalation. In one embodiment, the compositions provided herein may be administered by injecting directly to a tumor. In some embodiments, the compositions may be administered by intravenous injection or intravenous infusion. In certain embodiments, administration is systemic. In certain embodiments, administration is local.

### EXAMPLES

### Example 1

### Evaluation of a Mouse Partial Hepatectomy Liver Regeneration Model Treated with Cell Turnover Factors from AML-12 Mouse Hepatocyte Cells Exposed to Stress Conditions

### Methods

### Preparation of Conditioned Medium

AML-12 mouse hepatocytes were plated in 10 cm plates at 5.5 million cells per plate. The base medium for this cell line is DMEM:F12 Medium. To make the complete growth medium, the following components were added to the 500 mL of the base medium:

Supplemented with: 10% fetal bovine serum, 10 μg/ml insulin, 0.5 μg/ml transferrin, 5 ng/ml selenium, and 40 ng/ml dexamethasone. Cells were grown to at least 70% confluency overnight. Cells were washed with PBS 2X; 7 ml of serum-free media with ITS supplements without dexamethasone, and then treated with compounds. Cells were treated for 48h before conditioned medium was collected for processing.

Cells were collected with culture medium by pipetting and medium+cells collected in 50 mL conical tube. 100 uL of each sample was aliquoted for testing viability with CTG (add 100 uL of mix cells/medium to 96 wells plate+100 uL of CTG). The remainder of the sample was spun down (5 min@300 g). Supernatant were filtered with 0.45 uM filter and flow through was transferred to 3 kDA cutoff Amicon filtration units and spin @4000 g for 30-45 minutes. Column was washed with 5-10 ml PBS. Column was washed 3× total. All >3kDa supernatant fractions for each condition were collected. 1× PBS was added to each pooled sample to get a total volume of 12 ml/condition. 1 ml was aliquoted to a separate tube for protein quantification and proteomics. Remaining 11 ml was split into 2 tubes of 5.5 ml each (Dosing on Day 0 and Day 1). Samples were placed at −80° C. and used in the in vivo study.

TABLE 2
Conditioned Medium (CM) Treatments

| Sample name | Treatment |
|---|---|
| CM0 | Media alone |
| CM1 | Doxycycline at 4 μM |
| CM2 | Tunicamycin 0.5 μg/mL |
| CM3 | Venetoclax at 10 μM |
| CM4 | Freeze thaw |
| CM5 | Thioacetamide at 75 mM |
| CM6 | TNFalpha at 100 mg/mL, Birinapant at 10 |
| | μM, caspase inhibitor Q-VD-OPh (quinolyl- |
| | valyl-O-methylaspartyl-[2,6- |
| | difluorophenoxy]-methyl ketone) at 20 μM |
| CM7 | RSL3 at 10 μM |

### Evaluation of Mice Liver Regeneration Model

We evaluated the efficacy of the test compositions CM0, CM1, CM2, CM3, CM4, CM5, CM6 and CM7 in a mice liver regeneration model induced by partial hepatectomy. Hepatocyte growth factor (HGF)-plasmid was used as positive control. HGF-plasmid was diluted with saline to the concentration of 10.5 μg/ml. Test articles CM0, CM1, CM2, CM3, CM4, CM5, CM6 and CM7 were also diluted with saline (test articles: saline=1:4).

C57BL/6 mice (10 per treatment group) received test article 2 hours before surgery and received a 2nd dose 24 hours after surgery via hydrodynamic tail vein (HTV) injection. The injection volume was 8% of body weight. For the surgery, mice were anaesthetized with isofluorene (1-2.5%). The liver was exposed by an upper abdominal midline incision of 2-3 cm. The top of left lateral lobe and medial lobe of the liver was ligated with 4-0 silk suture placing the knot as close to the base of the lobe as possible. The tied lobe was removed just above the suture. Muscle and skin were sutured separately with 5-0 silk. For animals with the sham operation, the surgical procedure was the same except the liver was not ligated or transected.

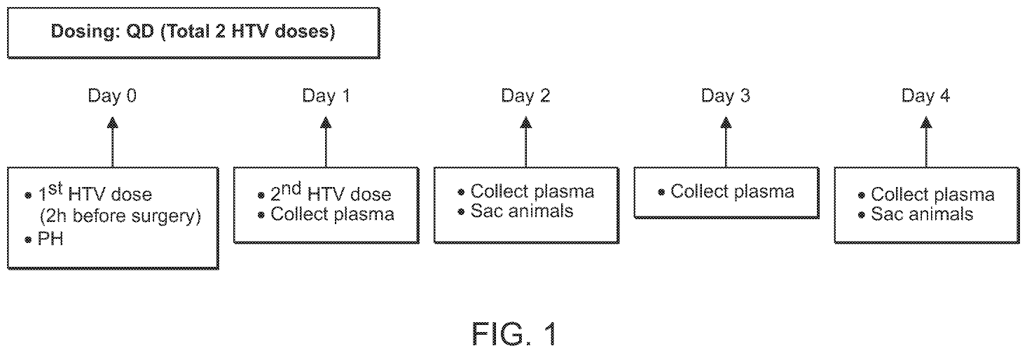

Plasma was collected on day 1, 2, 3, and 4. Animals were euthanized for liver collection on day 2 and day 4. FIG. 1 shows the dosing and sample collection schedule. Whole blood samples were collected into EDTA anticoagulation tubes and processed to plasma.

Liver samples were collected at the end of the experiment. Liver weights were recorded. Liver tissues from right lateral lobe were removed and stored in tubes containing 10% NBF for pathology evaluation and fixed for 24 hours. Samples were embedded in paraffin and processed to obtain 4 μm sections for staining with hematoxylin-eosin. The severity of necrosis and steatosis was evaluated semi-quantitatively using a predetermined score system (Table 3).

TABLE 3
Pathology evaluation criteria for liver necrosis and steatosis

| Grade | |
|---|---|
| | Necrosis |
| 0 | None |
| 1 | Few(<25% parenchymal) |
| 2 | Many (>25% parenchymal) |
| | Steatosis |
| 0 | <10% |
| 1 | 10%-20% |
| 2 | 20%-30% |
| 3 | >30% |

For immunohistochemical staining, 4 μm thick-sections were placed on slides and after overnight drying, the paraffin was removed by xylene. Then sections were placed in a graded ethanol series and immersed in distilled water. After heat-induced citrate antigen (pH 6.0) unmasking, sections were immersed in 3% hydrogen peroxide solution for 5 min. To avoid nonspecific staining, the sections were then incubated in blocking serum (DAKO#X0909) for 15 min at room temperature, followed by using primary rabbit polyclonal anti-PCNA antibodies (Abcam, #ab92552) or anti-Ki67 antibodies (Abcam, #ab15580) in dilution 1:600 or 1:1500 for 1 hour. Then secondary goat polyclonal antibodies conjugated to HRP (DAKO, #K4003) were added. For image analysis of positive cells, PCNA/Ki67 stained sections were used and scanned by Aperio CS2 Scan machine. HALO v2.3 workstation “IHC Nuclear vl” module was used.

In vivo animal data were expressed as mean ±SEM. Statistical analysis was performed using a One Way ANOVA followed by Dunnett's multiple comparison or a Two Way ANOVA followed by Bonferroni's multiple comparison. Non parametric tests like Mann-Whitney, etc were used when the N was too small or data did not follow Gaussian distribution. The difference was considered significant when p<0.05.

### Results

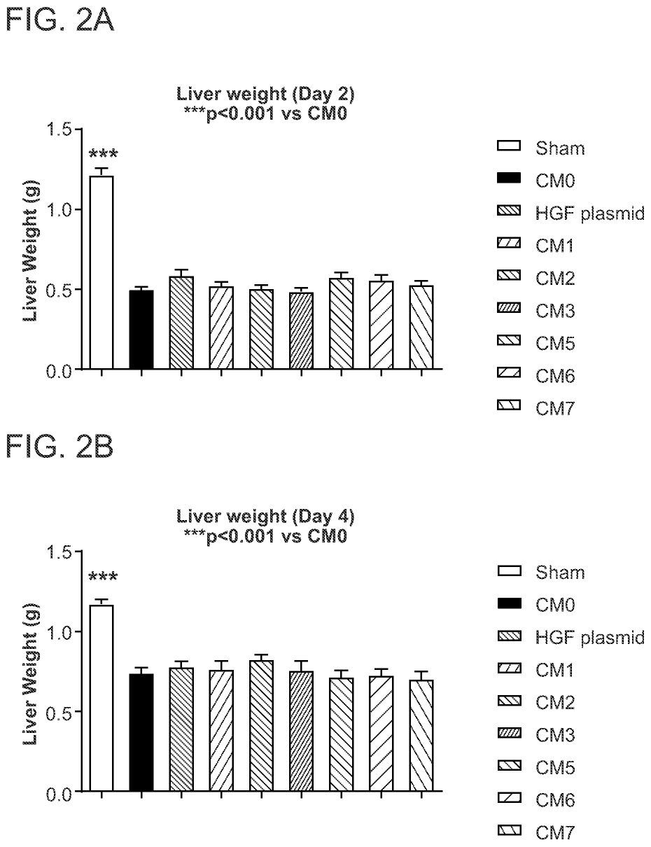

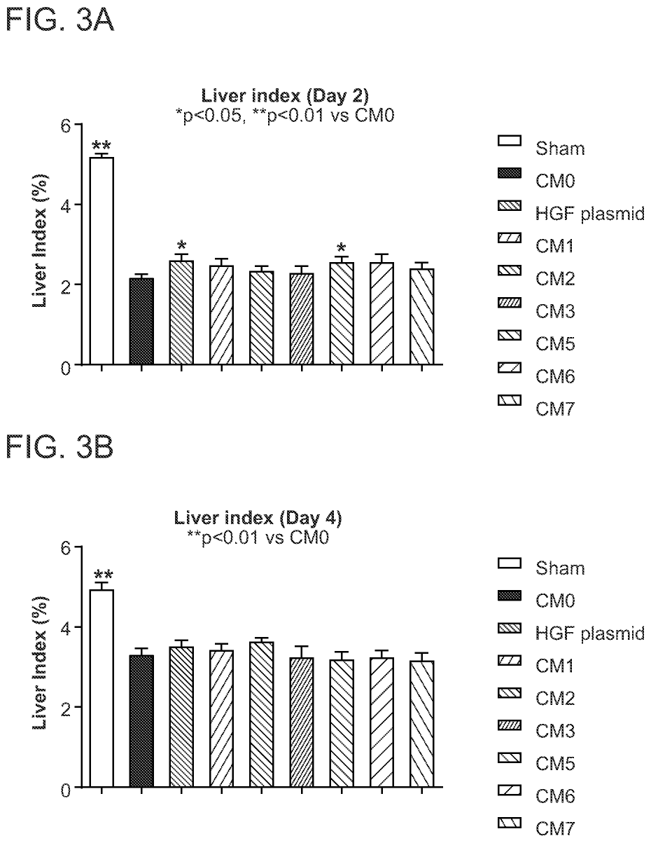

Liver weights and body weights were recorded on Days 2 and 4. The liver index was calculated by dividing the liver weight by the body weight. Surgery significantly decreased liver weight and liver index. Compared to CM0 group, treatment with HGF plasmid, CM1, CM2, CM3, CM5, CM6 and CM7 revealed no significant difference on liver weight on Day 2 and 4 (FIGS. 2A and 2B). Compared to CM0 group, treatment with HGF plasmid and CM5 significantly increased liver index on Day 2. CM1, CM2, CM3, CM6 and CM7 revealed no significant difference in liver index on Day 2 and 4 (FIGS. 3A and 3B).

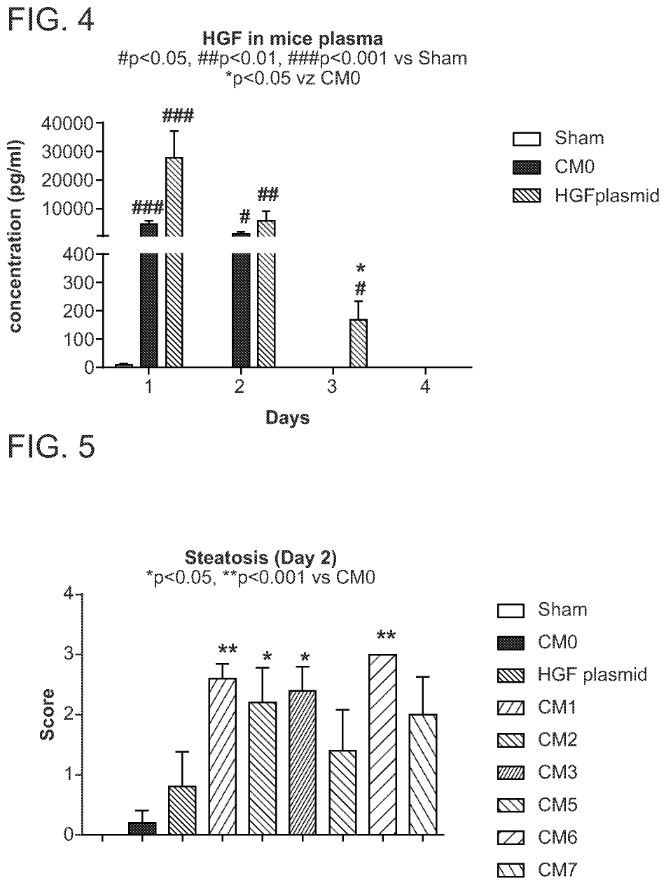

Partial hepatectomy treatment (CM0 and HGF group) significantly increased endogenous plasma HGF expression level on Day 1 and 2. HGF HTV injection further increased HGF expression through exogenous expression of HGF. Increased endogenous HGF expression decreased and cannot be detected on Day 3 for CM0 group. Compared to CM0 group, HGF plasmid treatment group significantly increased plasma HGF level on Day 3. Plasma HGF were not detected in Sham, CM0 and HGF plasmid groups on Day 4 via ELISA (FIG. 4).

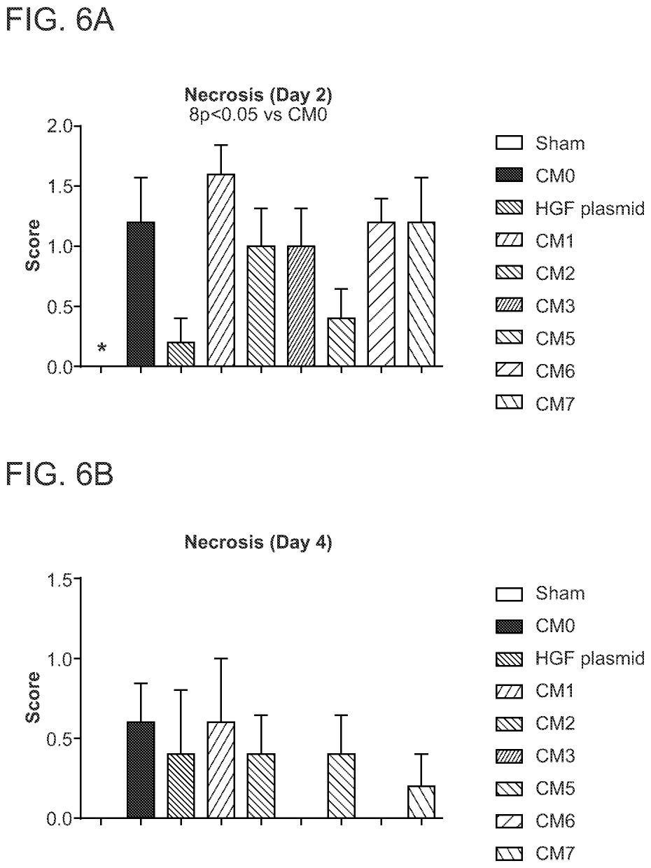

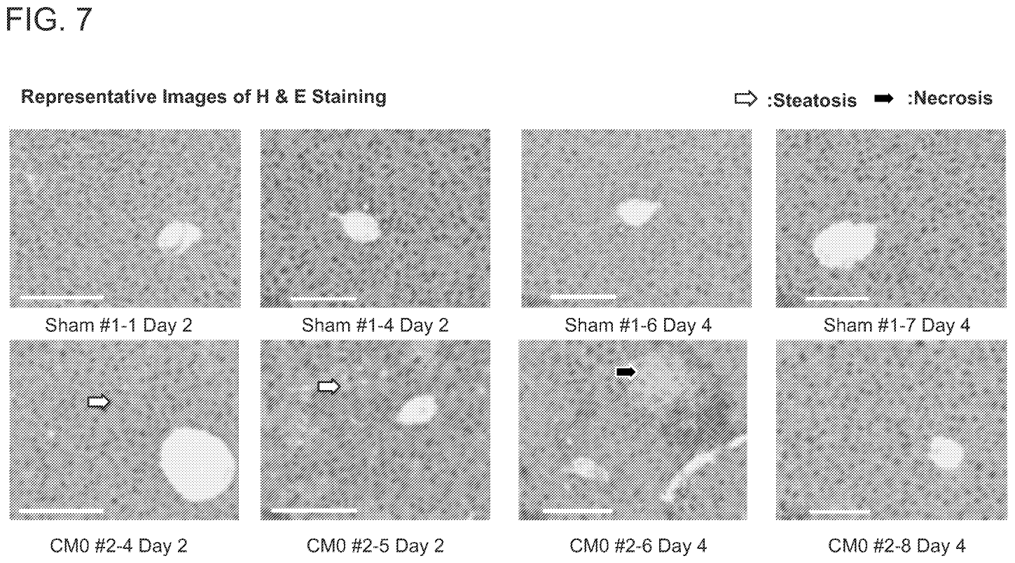

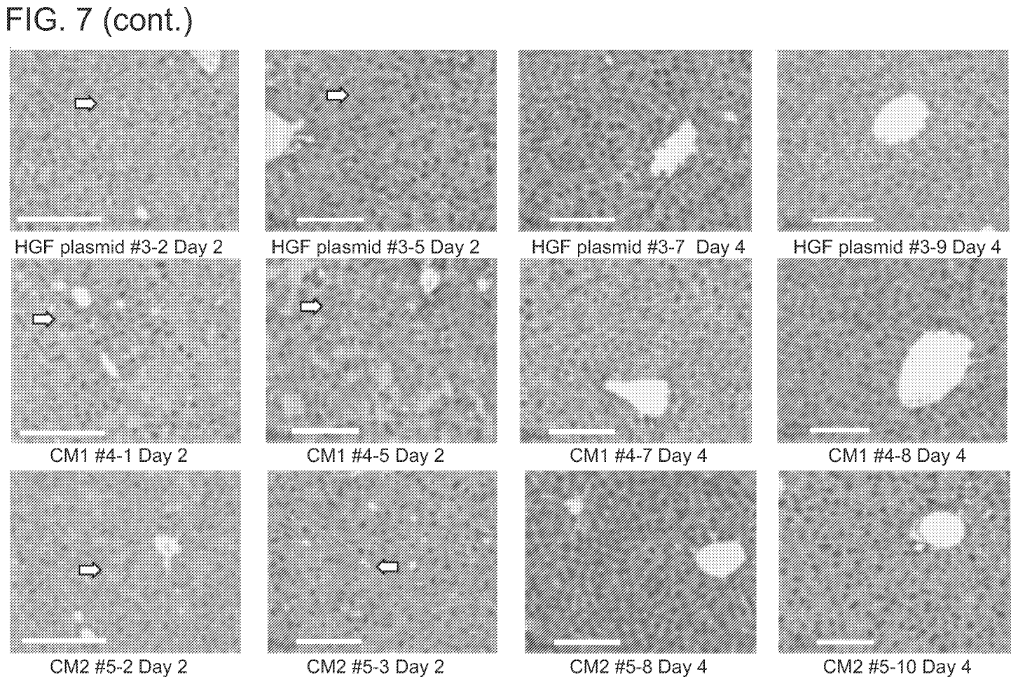

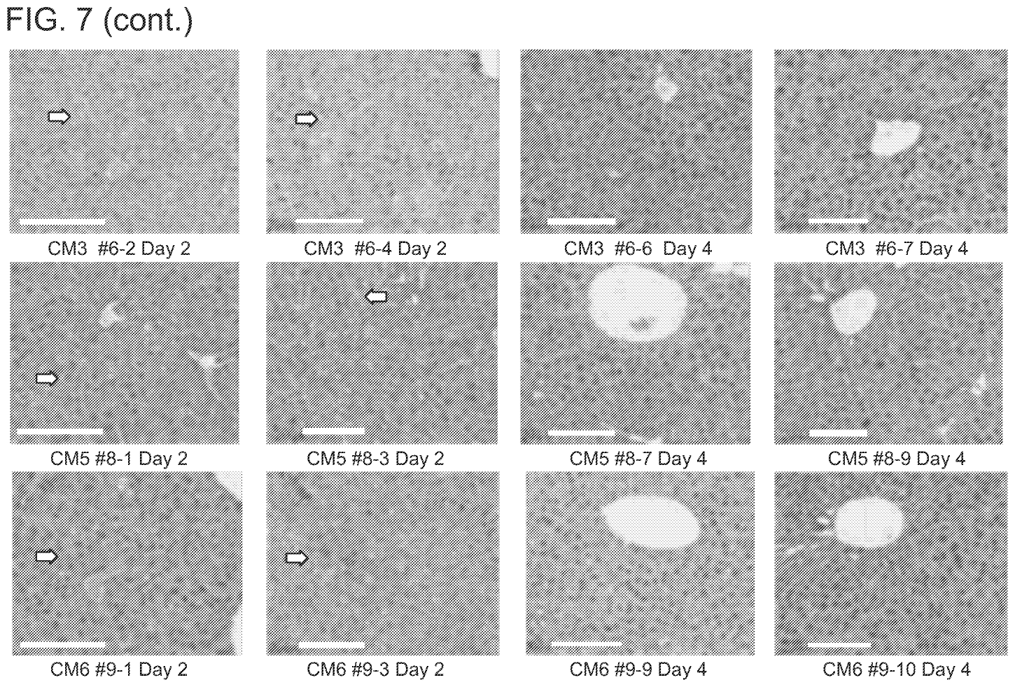

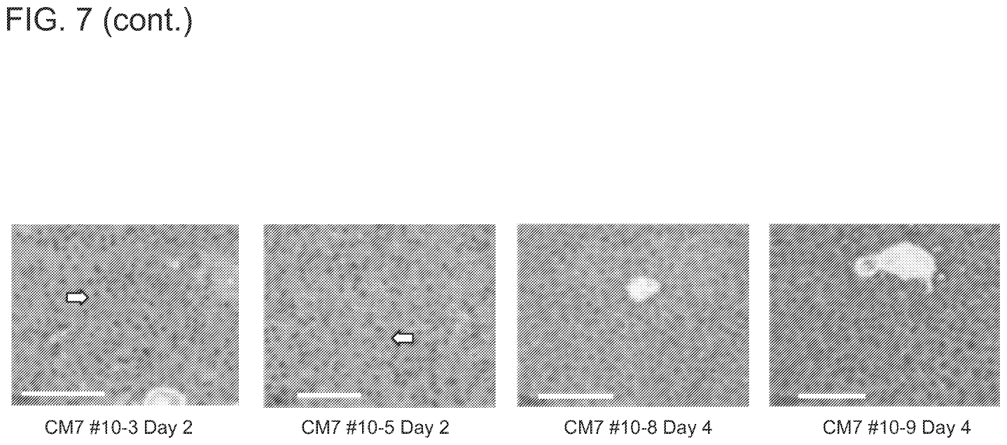

Compared to CM0 group, CM1, CM2, CM3 and CM6 treatment significantly increased the steatosis of liver on Day 2. HGF, CMS and CM7 trended to increase liver steatosis on Day 2. Liver steatosis was not observed on Day 4 (FIG. 5). Compared to Sham group, Partial hepatectomy treatment groups significantly increased liver necrosis. Compared to CM0 group, CM1, CM2, CM3, CM6 and CM7 treatment revealed no difference on liver necrosis on Day 2 and 4 while HGF plasmid and CM 5 treatment trended to decrease liver necrosis on Day 2 (FIGS. 6A, 6B and 7).

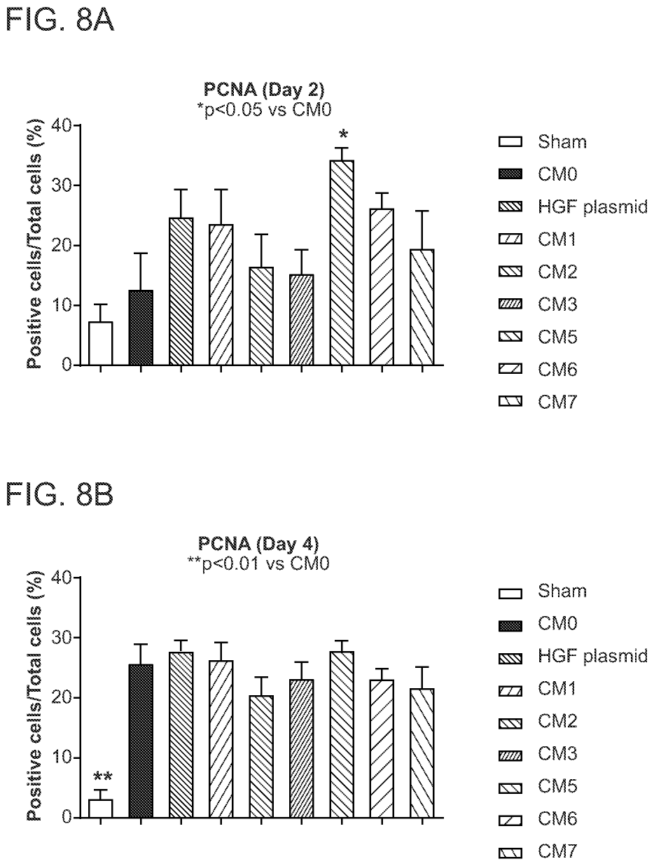

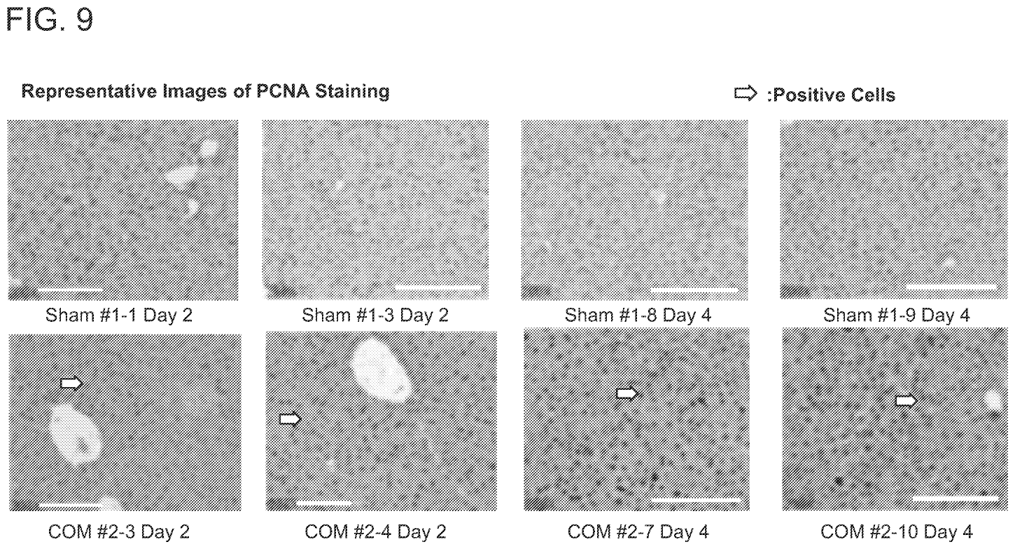

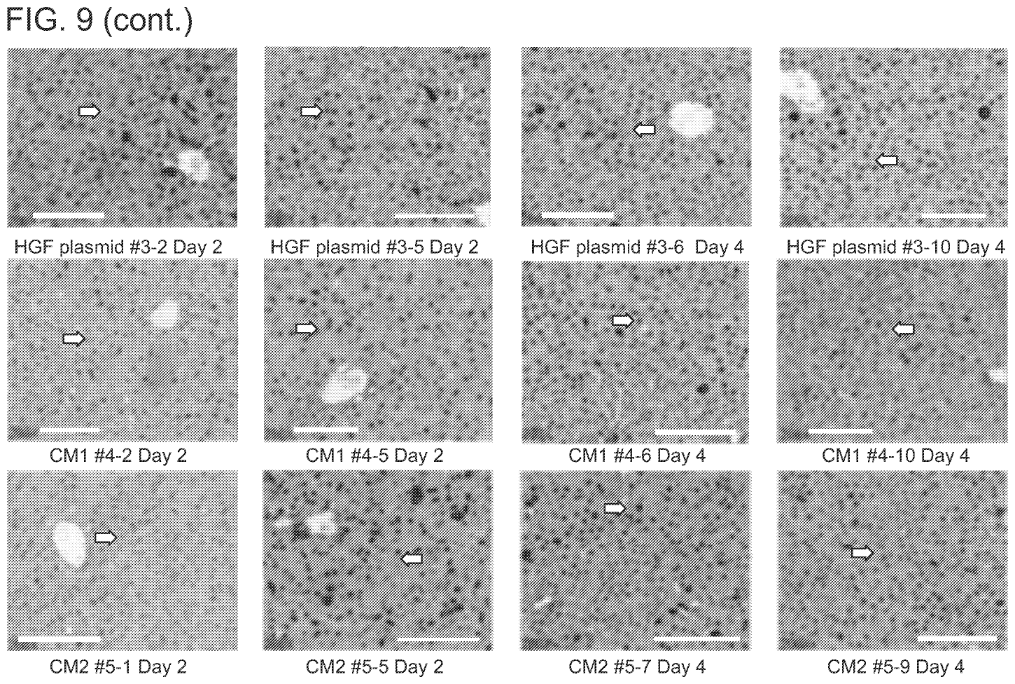

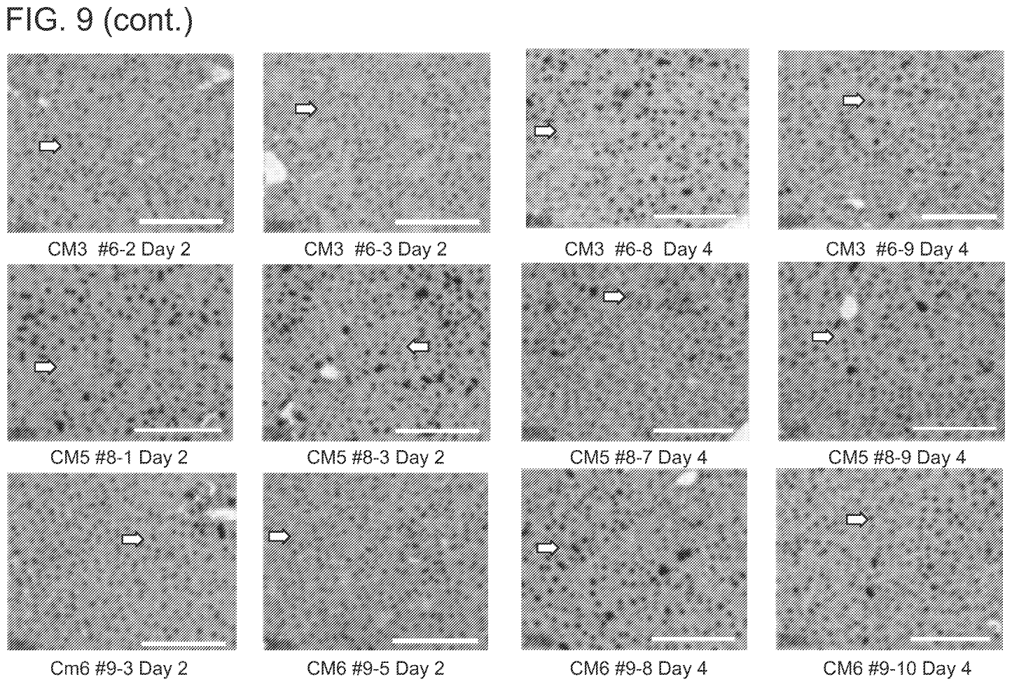

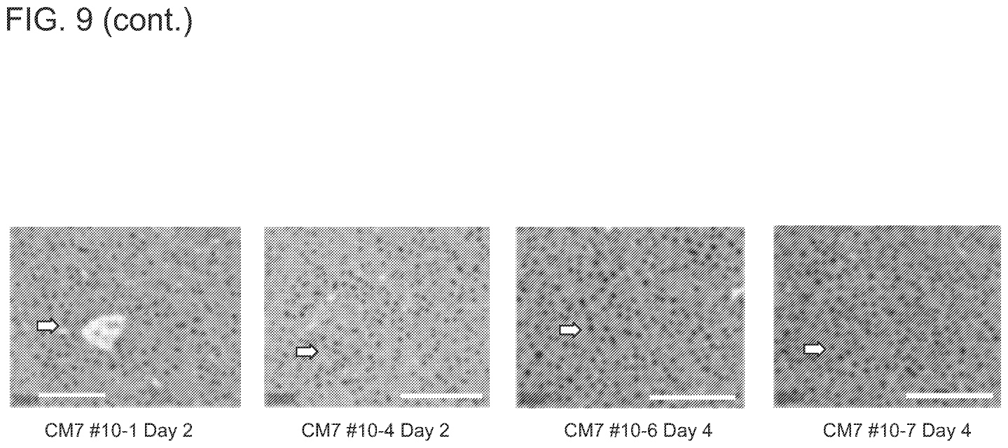

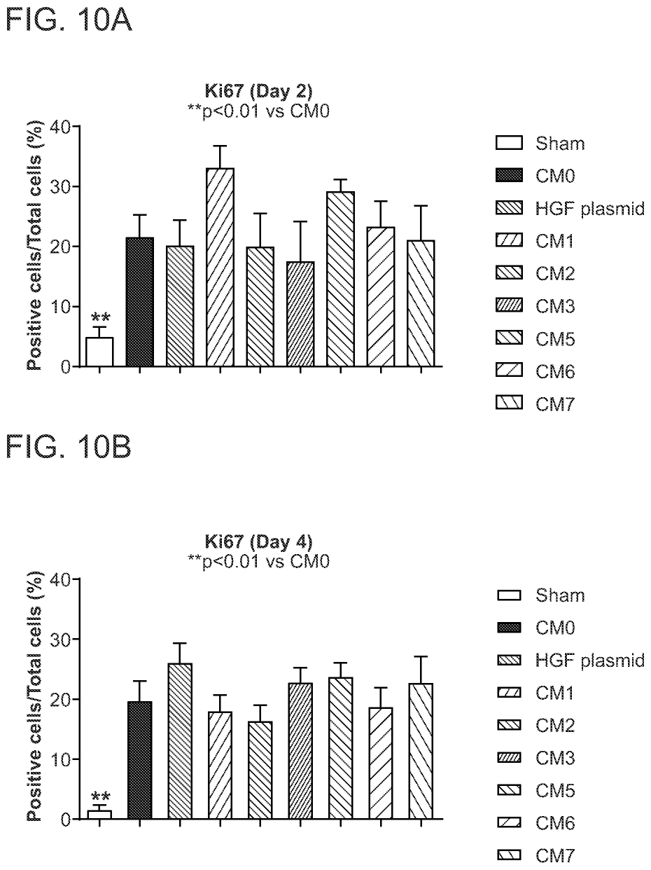

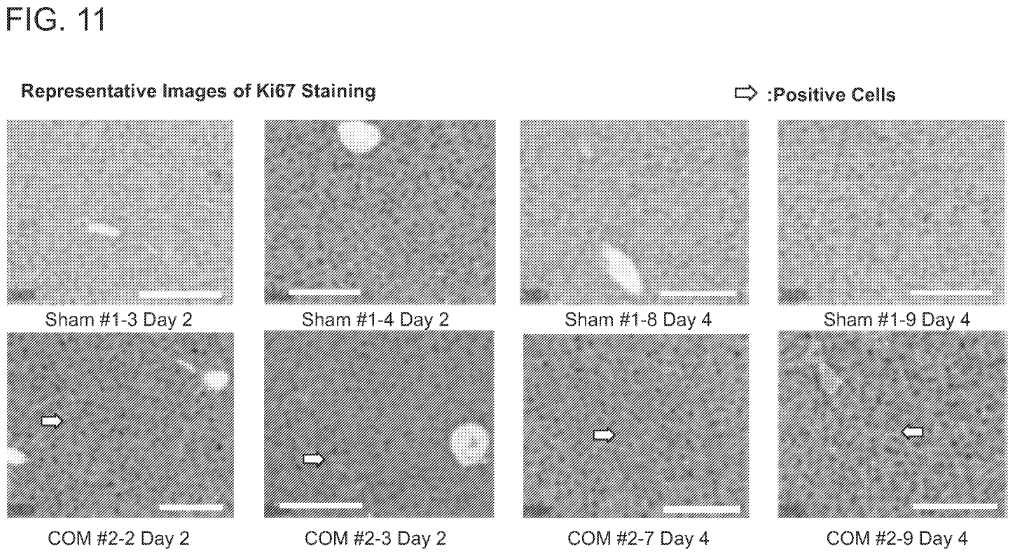

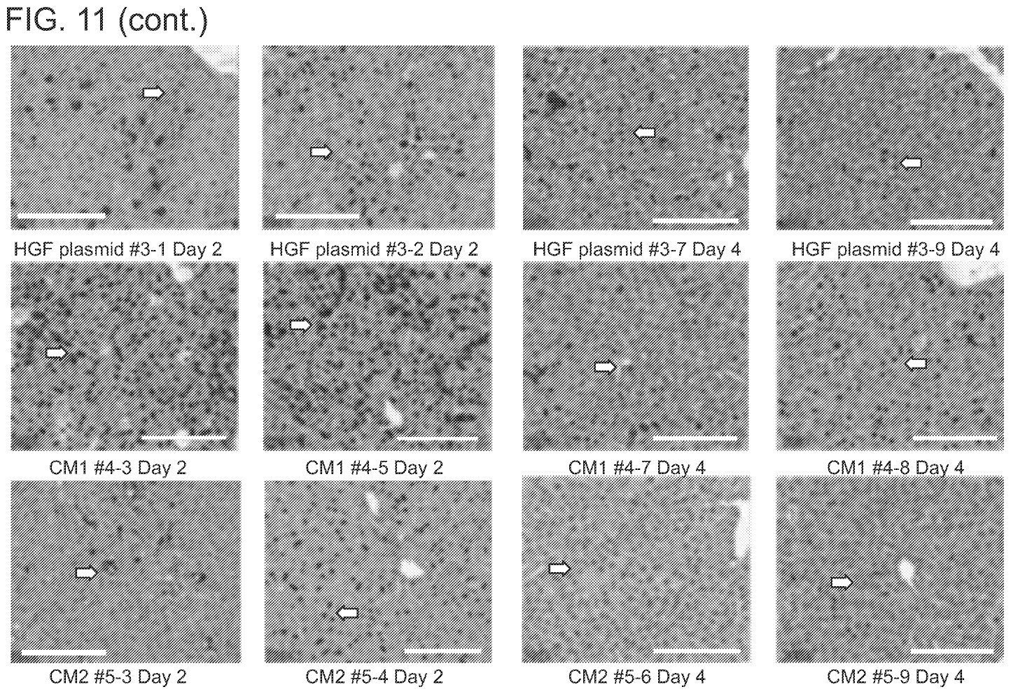

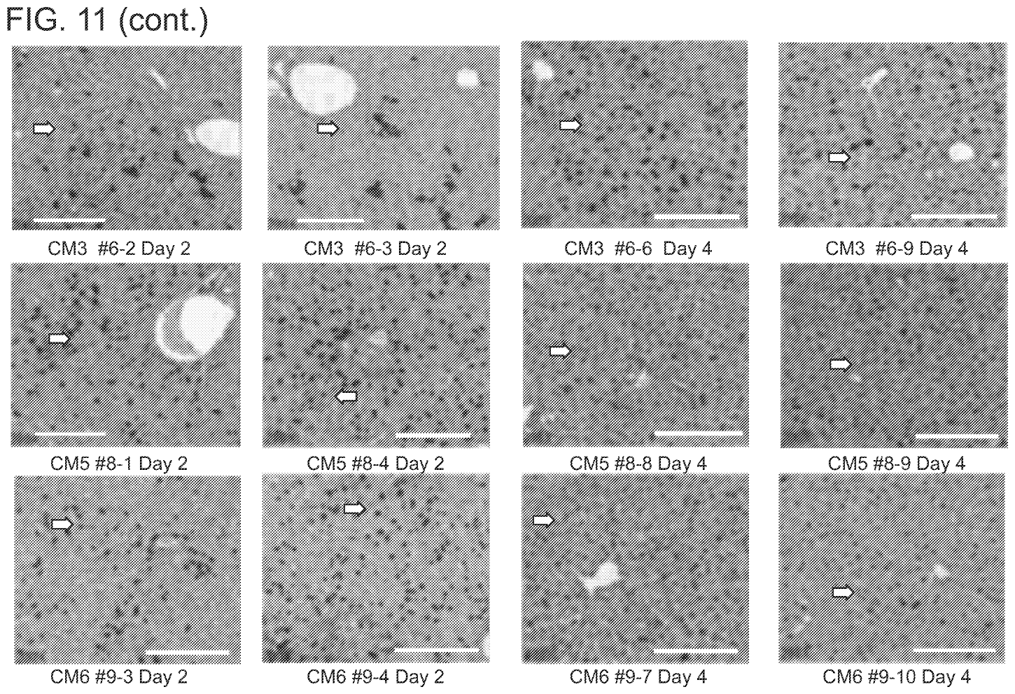

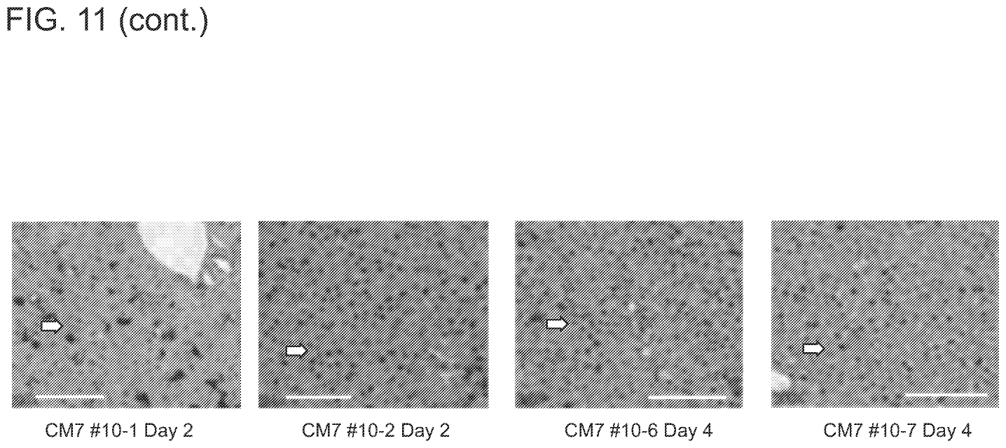

Cell proliferation markers Proliferating Cell Nuclear Antigen (PCNA) and Ki67 were observed in liver tissue after partial hepatectomy treatment (FIGS. 8A, 8B, 9, 10A, 10B and 11). Compared to Shamgroup, partial hepatectomy treatment trended to increase PCNA expression on Day 2 and significantly increased PCNA expression on Day 4. Compared to CM0 group, CMS treatment significantly increased PCNA expression on Day 2 while HGF plasmid, CM1, CM2, CM3, CM6 and CM7 treatment revealed no difference on PCNA expression on Days 2 and 4. These results indicate that CMS treatment increased proliferation of liver cells. Compared to Shamgroup, partial hepatectomy treatment significantly increased Ki67 expression on Day 2 and 4. Compared to CM0 group, CM1 and CMS treatments trended to increase Ki67 expression on Day 2 while HGF plasmid, CM2, CM3, CM6 and CM7 treatment revealed no difference on Ki67 expression on Day 2 and 4.

### Conclusions

Treatment of liver with cell turnover factors from liver cells exposed to a stress condition (i.e. treated with thioacetamide) induced liver tissue regeneration and lower levels of necroptosis in a mouse partial hepatectomy liver regeneration model treated.

CM0, CM1, CM2, CM3, CM4, CM6, CM7 (which are respectively, no treatment, doxorubicin, tunicamycin, venetoclax, freeze-thaw, TNF-alpha plus caspase inhibitor plus Birinapant, and RSL3) did not show a regenerative effect. CM5 treatment significantly increased liver index and liver cell PCNA expression on Day 2, indicating that CM5 treatment increased liver cell regeneration. In addition, CMS treatment trended to increase liver cell Ki67 expression and decrease liver necrosis on Day 2, further supporting the role of CMS treatment in increasing liver cell regeneration.

Equivalents

Those skilled in the art will recognize, or be able to ascertain using no more than routine experimentation, many equivalents to the specific embodiments and methods described herein. Such equivalents are intended to be encompassed by the scope of the following claims.

Incorporation by Reference

Each reference, patent, and patent application referred to in the instant application is hereby incorporated by reference in its entirety as if each reference were noted to be incorporated individually.

1. A method of increasing regeneration of a tissue in an organ of a mammalian subject, the method comprising administering to the subject a composition in an amount and for a time sufficient to increase regeneration of the tissue, wherein the composition is made by (a) exposing mammalian cells to a stress condition, and (b) collecting the exposed cells or conditioned media from the exposed cells to create the composition.

2. The method of claim 1, wherein the stress condition is a compound that induces endoplasmic reticlulum stress.

3. The method of claim 1, wherein the stress condition is selected from the group consisting of thioacetamide, tunicamycin, PhenolaTi, Zearalenone, Shiga toxin-2, carbon tetrachloride (CCL4) and acetaminophen.

4. The method of claim 1, wherein the stress condition is thioacetamide.

5. The method of any one of claims 1 to 4, wherein the mammalian cells exposed to the stress condition are of a type present in the tissue or organ.

6. The method of any one of claims 1 to 4, wherein the mammalian cells exposed to the stress condition are selected from the group consisting of liver cells, kidney cells, pancreatic cells, muscle cells, bone cells, cells of the intestinal lining, cardiac cells, lung cells, skin cells, neurons, cells of the central nervous system (CNS), epithelial cells, endothelial cells, fibroblasts, and immune cells.

7. The method of any one of claims 1 to 4, wherein the mammalian cells exposed to the stress condition are liver cells and the organ is liver.

8. The method of any one of claims 1 to 4, wherein the cells or the conditioned media are further fractioned to create the composition.

9. The method of any one of claims 1 to 4, wherein the mammalian cells exposed to the stress condition are autologous to the subject.

10. The method of any one of claims 1 to 4, wherein the mammalian subject is a human.

11. A method of increasing regeneration of a tissue in an organ of a subject, the method comprising administering to the subject a composition comprising one or more cell turnover factors produced by cells exposed to a stress condition, wherein the composition is administered to the subject in an amount sufficient to increase regeneration of the tissue relative to a subject that is not treated with the composition.

12. A method of delivering a composition comprising one or more cell turnover factors produced by cells exposed to a stress condition to a subject in need of increased tissue regeneration, the method comprising administering the composition to the subject.

13. The method of claim 1, 11 or 12, wherein the tissue is liver tissue and the subject has a disorder selected from the group consisting of chronic liver damage, alcoholic steatohepatitis (ASH), drug-induced liver injury, fulminant and late-onset hepatic failure (LOHF), fulminant hepatitis (FH), liver cirrhosis, liver fibrosis, fulminant hepatic failure (FHF), hepatitis B, and hepatitis C.

14. The method of claim 1, 11 or 12, wherein the tissue is kidney tissue and the subject has a disorder or condition selected from the group consisting of diabetes mellitus, rheumatoid arthritis, nephritic syndrome, nephrotic syndrome, hypertension nephropathy, polycystic kidney disease, progressive chronic kidney disease, chronic renal failure, Fabry disease, cystinosis, nephronophthisis, Alport's syndrome, reperfusion injury, acute kidney injury, kidney fibrosis and a kidney transplant.

15. The method of claim 1, 11 or 12, wherein the tissue is pancreatic tissue and the subject has a disorder selected from the group consisting of pancreatic cancer, diabetes mellitus, insulin resistance, hypoglycemia, hyperglycemia, lipase deficiency, cholecystokinin (CCK) deficiency, acute pancreatitis, chronic pancreatitis and hereditary pancreatitis.

16. The method of claim 1, 11 or 12, wherein the tissue is intestinal lining tissue and the subject has a disorder or condition selected from the group consisting of an inflammatory gastrointestinal disorder, Crohn's disease, inflammatory bowel disease (IBD), diverticulitis, parasitic infection, bacterial infection, a functional gastrointestinal disorder, ulcerative colitis (UC), and a surgical resection of the intestines.

17. The method of claim 1, 11 or 12, wherein the tissue is cardiac tissue and the subject has a cardiovascular disease selected from the group consisting of a myocardial infarction, heart failure, heart injury by ischemic event, and heart injury by a non-ischemic event.

18. The method of claim 1, 11 or 12, wherein the tissue is lung tissue and the subject has a disorder or condition selected from the group consisting of acute respiratory distress syndrome (ARDS), chronic obstructive pulmonary disease (COPD), lung cancer, bronchiolitis obliterans organizing pneumonia (BOOP), Coronavirus Disease 2019 (COVID-19), mesothelioma, cystic fibrosis, asthma, idiopathic pulmonary fibrosis, lung failure due to aging, pulmonary fibrosis, interstitial lung disease (ILD), pulmonary arterial hypertension and α1-antitrypsin disorder.

19. The method of claim 1, 11 or 12, wherein the tissue is muscle tissue and the subject has a disorder or condition selected from the group consisting of myositis, autoimmune disease, amyotrophic lateral sclerosis (ALS), sarcopenia, pediatric Charcot-Marie-Tooth disease, muscle loss due to aging, muscle strain from injury, muscle atrophy, muscular dystrophy, dermatomyositis, Guillain-Barré syndrome, multiple sclerosis, polio and polymyositis.

20. The method of claim 1, 11 or 12, wherein the composition is formulated to target an organ in which tissue regeneration is increased.

21. The method of claim 1, 11 or 12, wherein the composition is formulated to target liver, kidney, pancreas, muscle, intestinal lining, heart, or lung.

22. The method of claim 1, 11 or 12, wherein the composition is formulated to target liver.

23. The method of claim 1, 11 or 12, wherein the composition is formulated to target kidney.

24. The method of claim 1, 11 or 12, wherein the composition is administered to the subject in an amount sufficient to decrease necrosis in the tissue in which the tissue regeneration is increased, relative to a subject that is not treated with the composition.

25. The method of claim 1, 11 or 12, wherein the composition is administered to the subject in an amount sufficient to decrease steatosis in the tissue in which the tissue regeneration is increased, relative to a subject that is not treated with the composition.

26. The method of claim 1, 11 or 12, wherein the composition is administered to the subject in an amount sufficient to increase weight of the organ in which the tissue regeneration is increased, relative to a subject that is not treated with the composition.

27. The method of claim 1, 11 or 12, wherein the composition is administered to the subject in an amount sufficient to increase expression of one or more cell proliferation marker proteins in the tissue, relative to a subject that is not treated with the composition.

28. A method of increasing tissue regeneration in a transplanted organ or tissue, the method comprising treating an organ or tissue ex vivo with a composition comprising one or more cell turnover factors produced by cells exposed to a stress condition, wherein the organ or tissue is treated with the composition in an amount sufficient to increase tissue regeneration in the organ or tissue after transplantation to a subject relative to an organ or tissue that is not treated with the composition.

29. A method of treating a tissue, an organ, organoid or organ culture, the method comprising contacting a tissue, an organ, organoid or organ culture in vitro with a composition comprising one or more cell turnover factors produced by cells exposed to a stress condition.

30. A method of preparing an organ or tissue for transplantation to a subject, the method comprising treating an organ or tissue ex vivo with a composition comprising one or more cell turnover factors produced by cells exposed to a stress condition.

31. The method of claim 29 or 30, wherein, the organ or tissue is treated with the composition in an amount sufficient to increase tissue regeneration in the organ or tissue after transplantation to the subject relative to an organ or tissue that is not treated with the composition.

32. The method of any one of claims 28 to 30, wherein the organ or tissue is treated with the composition in an amount sufficient to increase survival of the subject after transplantation of the organ or tissue to the subject relative to a subject transplanted with an organ or tissue that is not treated with the composition.

33. The method of any one of claims 28 to 30, wherein the organ or tissue is treated with the composition in an amount sufficient to improve engraftment of the organ or tissue after transplantation to the subject relative to an organ or tissue that is not treated with the composition.

34. The method of any one of claims 28 to 30, wherein the organ or tissue is treated with the composition in an amount sufficient to prolong viability of the organ or tissue before transplantation to the subject relative to an organ or tissue that is not treated with the composition.

35. The method of any one of claims 1, 11, 12, and 28 to 30, wherein the stress condition comprises an abiotic stress, a biotic stress and/or a chemical stress.

36. The method of any one of claims 1, 11, 12, and 28 to 30, wherein the stress condition comprises an abiotic stress.

37. The method of claim 36, wherein the abiotic stress is selected from the group consisting of nutrient deprivation, heat, cold, radiation, hypoxia, osmotic pressure, and pH stress.

38. The method of any one of claims 1, 11, 12, and 28 to 30, wherein the stress condition comprises a biotic stress.

39. The method of claim 38, wherein the biotic stress is selected from the group consisting of a viral infection with a naturally occurring virus, a bacterial infection, and a fungal infection.

40. The method of any one of claims 1, 11, 12, and 28 to 30, wherein the stress condition induces the cells to undergo apoptosis.

41. The method of any one of claims 1, 11, 12, and 28 to 30, wherein the stress condition comprises a chemical stress.

42. The method of claim 41, wherein the chemical stress comprises contacting the cells with a compound that promotes production of cell turnover factors by the cells.

43. The method of any one of claims 1, 11, 12, and 28 to 30, wherein the cells exposed to a stress condition are allogeneic to the subject.

44. The method of any one of claims 11, 12, and 28 to 30, wherein the cells exposed to a stress condition are autologous to the subject.

45. The method of any one of claims 11, 12, and 28 to 30, wherein the cells exposed to a stress condition are selected from the group consisting of liver cells, kidney cells, pancreatic cells, muscle cells, bone cells, cells of the intestinal lining, cardiac cells, lung cells, skin cells, neurons, cells of the central nervous system (CNS), epithelial cells, endothelial cells, fibroblasts, and immune cells.

46. The method of claim 45, wherein the epithelial cells, endothelial cells, fibroblasts or immune cells are from the liver, kidney, pancreas, muscle, bone, intestinal lining, heart, lung, skin, or the central nervous system.

47. The method of any one of claims 1, 11, 12, and 28 to 30, wherein the cells exposed to a stress condition are from the same type of tissue in which the tissue regeneration occurs.

48. The method of claim 47, wherein the cells exposed to a stress condition are liver cells, and the tissue in which the tissue regeneration occurs is liver tissue; the cells exposed to a stress condition are kidney cells and the tissue in which the tissue regeneration occurs is kidney tissue; the cells exposed to a stress condition are lung cells and the tissue in which the tissue regeneration occurs is lung tissue; the cells exposed to a stress condition are muscle cells and the tissue in which the tissue regeneration occurs is muscle tissue; the cells exposed to a stress condition are bone cells and the tissue in which the tissue regeneration occurs is bone tissue; the cells exposed to a stress condition are pancreatic cells and the tissue in which the tissue regeneration occurs is pancreatic tissue; the cells exposed to a stress condition are cardiac cells and the tissue in which the tissue regeneration occurs is cardiac tissue; the cells exposed to a stress condition are cells of the intestinal lining and the tissue in which the tissue regeneration occurs is the lining of the intestine; the cells exposed to a stress condition are skin cells and the tissue in which the tissue regeneration occurs is skin tissue; the cells exposed to a stress condition are cells of the CNS and the tissue in which the tissue regeneration occurs is CNS tissue; the cells exposed to a stress condition are epithelial cells and the tissue in which the tissue regeneration occurs is epithelium; or the cells exposed to a stress condition are endothelial cells and the tissue in which the tissue regeneration occurs is endothelium.

49. The method of any one of claims 1, 11, 12, and 28 to 30, wherein the cells exposed to a stress condition are not from the same type of tissue in which the tissue regeneration occurs.

50. The method of any one of claims 1, 11, 12, and 28 to 30, wherein the cells exposed to a stress condition are cancer cells.

51. The method of claim 50, wherein the cancer cells are immortalized.

52. The method of claim 50, wherein the cancer cells are primary cells isolated from a subject.

53. The method of any one of claims 1, 11, 12, and 28 to 30, wherein the composition does not comprise intact cells.

54. The method of any one of claims 1 to 53, wherein the composition comprises a cell-free extract prepared from the cells exposed to a stress condition.

55. The method of any one of claims 1, 11, 12, and 28 to 30, wherein the composition comprises conditioned media from the cells exposed to a stress condition.

56. The method of claim 55, wherein the composition comprises a functional fraction of the conditioned media.

57. The method of claim 56, wherein the functional fraction is prepared by isolating molecules based on molecular weight from the conditioned media.

58. The method of any one of claims 1 to 57, wherein the composition comprises one or more cell turnover factors isolated from the cells.

59. The method of claim 58, wherein the one or more cell turnover factors are purified or partially purified.

60. The method of any one of claims 1 to 59, wherein the composition comprises at least ten cell turnover factors.

61. The method of any one of claims 1 to 59, wherein the composition comprises at least two cell turnover factors.

62. The method of any one of claims 1 to 59, wherein the composition comprises only one cell turnover factor.

63. The method of any one of claims 1, 11 and 28 to 30, wherein the organ is a solid organ.

64. The method of any one of claims 1, 11 and 28 to 30, wherein the organ is selected from liver, kidney, pancreas, muscle, intestinal lining, heart, and lung.

65. The method of any one of claims 1, 11, 28 to 20, 63 and 64, wherein the organ has an injury.

66. The method of claim 65, wherein the injury is caused by a drug, a toxin, viral infection, or surgery to the organ.

67. The method of claim 65 or 66, wherein the composition is administered to the subject after the organ injury.

68. The method of any one of claims 1, 11 and 63 to 67, wherein the subject is in need of surgery to the organ.

69. The method of claim 68, wherein the composition is administered to the subject before surgery to the organ.

70. The method of claim 68 or 69, wherein the composition is administered to the subject after surgery to the organ.

71. The method of any one of claims 67 to 70, wherein the surgery comprises cancer resection involving the organ.

72. The method of any one of claims 1, 11, 12 and 28 to 30, wherein the tissue is selected from the group consisting of liver tissue, kidney tissue, pancreas tissue, muscle tissue, intestinal lining, cardiac tissue and lung tissue.

73. The method of any one of claims 1, 11, 12 and 28 to 30, wherein the method further comprises a step of preparing the composition comprising the one or more cell turnover factors.

74. The method of claim 73, wherein the step of preparing the composition comprises exposing the cells to the stress condition.

75. A method of increasing regeneration of a tissue in an organ of a subject, the method comprising administering to the subject a composition comprising a compound that induces cell turnover of a target cell in the subject, wherein the composition is administered in an amount sufficient to increase regeneration of the tissue relative to a subject that is not treated with the composition.

76. The method of claim 75, wherein the compound induces endoplasmic reticulum (ER) stress in the target cell.

77. The method of claim 76, wherein the ER stress comprises an unfolded protein response.

78. The method of claim 75, wherein the compound induces apoptosis in the target cell.

79. The method of claim 75, wherein the compound induces production of reactive oxygen species (ROS) in the target cell.

80. The method of claim 75, wherein the production of ROS in the target cell is at a level sufficient to induce death of the target cell.

81. The method of any one of claims 75 to 80, wherein the compound is a small molecule.

82. The method of any one of claims 75 to 80, wherein the compound is a protein.

83. The method of any one of claims 75 to 82, wherein the compound is targeted to the organ in which tissue regeneration is increased.

84. The method of any one of claims 75 to 83, wherein the compound is targeted to liver, kidney, pancreas, muscle, bone, intestinal lining, heart, or lung.

85. The method of claim 75, wherein the compound is conjugated to an antibody targeted to the organ in which tissue regeneration is increased.

86. The method of claim 85, wherein the antibody specifically binds to a tissue-specific antigen of the organ in which tissue regeneration is increased.

87. The method of claim 85 or 86, wherein the antibody is targeted to liver.

88. The method of any one of claims 75 to 87, wherein the compound is a toxin.

89. The method of claim 88, wherein the compound is selected from the group consisting of thioacetamide, tunicamycin, PhenolaTi, Zearalenone, Shiga toxin-2, carbon tetrachloride (CCL4) and acetaminophen.

90. The method of claim 75, wherein the compound is a chemotherapeutic agent.

91. The method of any one claims 75 to 90, wherein the compound is not a compound that induces iron-dependent cellular disassembly.

92. The method of any one of claims 75 to 91, wherein the target cell is a liver cell and the tissue is liver tissue; the target cell is a kidney cell and the tissue is kidney tissue; the target cell is a lung cell and the tissue is lung tissue; the target cell is a muscle cell and the tissue is muscle tissue; the target cell is a bone cell and the tissue is bone tissue; the target cell is a pancreatic cell and the tissue is pancreatic tissue; the target cell is a cardiac cell and the tissue is cardiac tissue; the target cell is a cell of the intestinal lining and the tissue is the lining of the intestine; the target cell is a skin cell and the tissue is skin tissue; the target cell is a cell of the CNS and the tissue is CNS tissue; the target cell is an epithelial cell and the tissue is epithelium; or the target cell is an endothelial cell and the tissue is endothelium.

93. The method of any one of claims 75 to 91, wherein the target cell is in the organ or tissue in which tissue regeneration is increased.

94. The method of any one of claims 75 to 91, wherein the target cell is in a tissue adjacent to the organ or tissue in which tissue regeneration is increased.

95. The method of claim 75, wherein the target cell is selected from the group consisting of an epithelial cell, a fibroblast, an endothelial cell and an immune cell.

96. The method of claim 95, wherein the immune cell is a monocyte or a macrophage.
